SEQUENCE LISTING (49 sequences)

SEQ ID NO: 1 - protein, 6 aa, Homo sapiens
RRPYIL

SEQ ID NO: 2 - protein, 6 aa, Artificial Sequence
  completely synthesized amino acid sequence
XKPWXL

SEQ ID NO: 3 - protein, 6 aa, Artificial Sequence
  completely synthesized amino acid sequence
XKPXXL

SEQ ID NO: 4 - protein, 6 aa, Artificial Sequence
  completely synthesized amino acid sequence
XRPYXL

SEQ ID NO: 5 - protein, 6 aa, Artificial Sequence
  completely synthesized amino acid sequence
XRPYXL

SEQ ID NO: 6 - protein, 6 aa, Artificial Sequence
  completely synthesized amino acid sequence
XRPYXL

SEQ ID NO: 7 - protein, 6 aa, Artificial Sequence
  completely synthesized amino acid sequence
XRPYXL

SEQ ID NO: 8 - protein, 6 aa, Artificial Sequence
  completely synthesized amino acid sequence
XRPYXL

SEQ ID NO: 9 - protein, 6 aa, Artificial Sequence
  completely synthesized amino acid sequence
XRPYXL

SEQ ID NO: 10 - protein, 6 aa, Artificial Sequence
  completely synthesized amino acid sequence
XRPYXL

SEQ ID NO: 11 - protein, 6 aa, Artificial Sequence
  completely synthesized amino acid sequence
XRPYXL

SEQ ID NO: 12 - protein, 6 aa, Artificial Sequence
  completely synthesized amino acid sequence
XRPYXL

SEQ ID NO: 13 - protein, 6 aa, Artificial Sequence
  completely synthesized amino acid sequence
XRPYXL

SEQ ID NO: 14 - protein, 6 aa, Artificial Sequence
  completely synthesized amino acid sequence
XRPYXL

SEQ ID NO: 15 - protein, 6 aa, Artificial Sequence
  completely synthesized amino acid sequence
XRPYXL

SEQ ID NO: 16 - protein, 6 aa, Artificial Sequence
  completely synthesized amino acid sequence
XRPYXL

SEQ ID NO: 17 - protein, 6 aa, Artificial Sequence
  completely synthesized amino acid sequence
XRPYXL

SEQ ID NO: 18 - protein, 6 aa, Artificial Sequence
  completely synthesized amino acid sequence
XKPWXL

SEQ ID NO: 19 - protein, 6 aa, Artificial Sequence
  completely synthesized amino acid sequence
XKPWXL

SEQ ID NO: 20 - protein, 6 aa, Artificial Sequence
  completely synthesized amino acid sequence
XRPWXL

SEQ ID NO: 21 - protein, 6 aa, Artificial Sequence
  completely synthesized amino acid sequence
XRPWXL

SEQ ID NO: 22 - protein, 6 aa, Artificial Sequence
  completely synthesized amino acid sequence
XRPWXL

SEQ ID NO: 23 - protein, 6 aa, Artificial Sequence
  completely synthesized amino acid sequence
XRPWXL

SEQ ID NO: 24 - protein, 6 aa, Artificial Sequence
  completely synthesized amino acid sequence
XRPWXL

SEQ ID NO: 25 - protein, 6 aa, Artificial Sequence
  completely synthesized amino acid sequence
XRPWXL

SEQ ID NO: 26 - protein, 6 aa, Artificial Sequence
  completely synthesized amino acid sequence
RRPXIL

SEQ ID NO: 27 - protein, 6 aa, Artificial Sequence
  completely synthesized amino acid sequence
RRPXIL

SEQ ID NO: 28 - protein, 6 aa, Artificial Sequence
  completely synthesized amino acid sequence
RRPXXL

SEQ ID NO: 29 - protein, 6 aa, Artificial Sequence
  completely synthesized amino acid sequence
XRPXXL

SEQ ID NO: 30 - protein, 6 aa, Artificial Sequence
  completely synthesized amino acid sequence
XRPXXL

SEQ ID NO: 31 - protein, 6 aa, Artificial Sequence
  completely synthesized amino acid sequence
XRPXIL

SEQ ID NO: 32 - protein, 6 aa, Artificial Sequence
  completely synthesized amino acid sequence
XKPXXL

SEQ ID NO: 33 - protein, 6 aa, Artificial Sequence
  completely synthesized amino acid sequence
XRPXXL

SEQ ID NO: 34 - protein, 6 aa, Artificial Sequence
  completely synthesized amino acid sequence
XXPXXL

SEQ ID NO: 35 - protein, 5 aa, Artificial Sequence
  completely synthesized amino acid sequence
XPXXL

SEQ ID NO: 36 - protein, 5 aa, Artificial Sequence
  completely synthesized amino acid sequence
XPXIL

SEQ ID NO: 37 - protein, 5 aa, Artificial Sequence
  completely synthesized amino acid sequence
XPXXL

SEQ ID NO: 38 - protein, 5 aa, Artificial Sequence
  completely synthesized amino acid sequence
XPXIL

SEQ ID NO: 39 - protein, 6 aa, Artificial Sequence
  completely synthesized amino acid sequence
RXPXIL

SEQ ID NO: 40 - protein, 6 aa, Artificial Sequence
  completely synthesized amino acid sequence
RXPXXL

SEQ ID NO: 41 - protein, 8 aa, Artificial Sequence
  completely synthesized amino acid sequence
DRVXIHPF

SEQ ID NO: 42 - protein, 9 aa, Artificial Sequence
  completely synthesized amino acid sequence
RPPGXSPFR

SEQ ID NO: 43 - protein, 5 aa, Artificial Sequence
  completely synthesized amino acid sequence
XGGFL

SEQ ID NO: 44 - protein, 5 aa, Artificial Sequence
  completely synthesized amino acid sequence
RPYIL

SEQ ID NO: 45 - protein, 5 aa, Artificial Sequence
  completely synthesized amino acid sequence
RPYXL

SEQ ID NO: 46 - protein, 5 aa, Artificial Sequence
  completely synthesized amino acid sequence
KPYXL

SEQ ID NO: 47 - protein, 5 aa, Artificial Sequence
  completely synthesized amino acid sequence
KPWXL

SEQ ID NO: 48 - protein, 5 aa, Artificial Sequence
  completely synthesized amino acid sequence
RPWXL

SEQ ID NO: 49 - protein, 13 aa, Homo sapiens
  MOD_RES (1)..(1): cis-1-(5-oxo-L-prolyl)
XLYENKPRRPYIL

(19) United States
(12) Patent Application Publication
(10) Pub. No.: US 2012/0142601 A1
(43) Pub. Date: Jun. 7, 2012

(54) Modified Peptides and Uses Thereof

(75) Inventors: George Walter Koszalka, Chapel Hill, NC (US); Justin Orion Brower, Cary, NC (US)

(73) Assignee: MUSC Foundation for Research Development

(21) Appl. No.: 13/187,254

(22) Filed: Jul. 20, 2011

Related U.S. Application Data
(63) Continuation of application No. PCT/US10/21826, filed on Jan. 22, 2010.
(60) Provisional application No. 61/146,923, filed on Jan. 23, 2009.

(51) Int. Cl. A61K 38/08; A61P 25/18; A61P 25/00; A61P 43/00; A61P 29/00

(52) U.S. Cl. 514/17.5; 514/21.8; 514/18.3; 514/21.7; 514/21.6

(57) ABSTRACT

The invention includes compounds that are useful in treating perioperative shivering or temperature spiking, lowering body temperature, treating psychosis or treating pain. The invention also includes methods for treating perioperative shivering or temperature spiking, lowering body temperature, treating psychosis or treating pain in a subject in need thereof.

### BACKGROUND OF INVENTION

Non-natural amino acids may influence the structural and biological activity of peptides in which they are incorporated (Moore et al., 1978, Can. J. Biochem. 56:315). As an example, homolysine and homoarginine analogs of vasopressin have reduced antidiuretic effect (Lindeberg et al., 1977, Int, J. Peptide Protein Res. 10:240).

Naturally occurring endogenous peptides are ideal leads for drug candidates because they promote and regulate biological processes. However, the endogenous peptides themselves are inherently poor drug candidates because they most often exert localized effects and are rapidly degraded within the body. In addition, most peptides are unable to cross biological membranes, including the small intestine and blood brain barrier (BBB). Furthermore, peptides often bind to more than one receptor or receptor subtype, rarely showing the selectivity required of a viable drug candidate. Therefore, for a peptide to become a viable drug candidate, improvements in blood stability, receptor selectivity and barrier crossing should be made without eliminating inherent binding affinity.

Numerous strategies have been developed to improve peptide stability, including N- and C-terminal modifications, amide backbone modifications, and introduction of conformational constraints in the peptidic chain. In order to improve the membrane permeability of a biologically active peptide, the peptide may be conjugated to a permeabilizing agent that allows the peptide to cross biological membranes; the peptide is then release from the conjugate by endogenous enzymes. While each of these strategies has been used to improve peptides as drug candidates, a universal solution for creating stable and receptor-selective peptides that cross biological barriers has not been identified.

Neurotensin (NT) is a neuropeptide with multiple biological activities that has been used as a starting point for the design of novel therapeutic agents. NT was first isolated from bovine hypothalami as a hypotensive peptide in 1973. Since then, NT has been shown to have physiological effects in the central nervous system (CNS) and the periphery. Hypothermia, antinociception, attenuation of d-amphetamine-induced hyperlocomotion and potentiation of barbiturate-induced sedation are promoted by direct injection of NT into the brain. Peripherally, NT acts as a hormone to induce hypotension and decrease gastric acid secretion. Structurally, NT is a linear tridecapeptide with the following sequence: pGlu-L-Leu-L-Tyr-L-Glu-L-Asn-L-Lys-L-Pro-L-Arg-L-Arg-L-Pro-L-Tyr-L-Ile-L-Leu-OH (SEQ ID NO:49), where pGlu is the cyclic analogue of the natural L-glutamate amino acid. Early in the history of NT research, the C-terminal hexapeptide NT(8-13), with sequence L-Arg⁸-L-Arg⁹-L-Pro¹⁰-L-Tyr¹¹-L-Ile¹²-L-Leu¹³ (SEQ ID NO:1), was found to be equipotent at producing the physiological effects of NT in vitro and in vivo.

An NT receptor (NTR₁) was first isolated from rat brain in 1990 (Tanaka et al., 1990, Neuron. 4:847-854). Since then, human NTR₁ has been successfully cloned and expressed. Both the rat and human receptors are classic G-protein coupled receptors containing seven transmembrane (7™) domains and share 84% homology. Second messenger systems, including cGMP production, calcium mobilization and phosphatidyl-inositol turnover, are triggered upon NTR₁ activation. The mRNA for NTR₁ is expressed in both rat and human brain and intestine.

A second NT receptor (NTR₂) with a substantially lower affinity for NT than NTR₁ (K_{d}˜2.5 and 0.5 nM respectively) also has been identified in rat and human brain (Hermans & Maloteaux, 1998, Pharmacol. Ther. 79:89-104; Vincent, 1995, Cell Mol. Neurobiol. 15:501-512; Chalon et al., 1996, FEBS Lett. 386:91-94). NTR₂ is also a 7™/G-protein coupled receptor, yet has a shorter N-terminal extracellular tail and a longer third intra-cytoplasmic loop compared to NTR₁.

A third receptor (NTR₃) was cloned from a human brain cDNA library and found to be identical to the previously cloned gp95/sortilin. NTR₃ is a non-G-protein coupled sorting protein having only a single transmembrane region.

NT appears to be involved in the pathophysiology of schizophrenia. Advances in the dopamine theory of schizophrenia indicate that a flaw in the convergence of various neural circuits on the mesolimbic dopamine system is responsible for the development of the disorder. The anatomical positioning of the NT system is such that it interacts with the glutaminergic, dopaminergic, GABAergic, and serotonergic systems within the brain. In particular, the NT and dopamine systems are closely related within the nucleus accumbens, the area of the brain believed to be responsible for delusions and hallucinations. NTR₁ receptors are dense in the ventral tegmental area, a brain region closely associated with the neuronal systems described above. Almost 90% of NT receptors are located on dopaminergic neurons and over 80% of dopamine neurons in the brain express NTR₁. Co-localization of the NT system with brain regions implicated in schizophrenia also implies its involvement.

Since NT was hypothesized as an “endogenous neuroleptic” and NT(8-13) was identified as its active fragment, efforts have been made to develop NT(8-13) analogues as potential antipsychotics. Analogues of NT(8-13) showed promise as antipsychotic drugs (see, for example, U.S. Pat. Nos. 6,214,790; 6,765,099; 6,921,805; and 7,098,307, all of which incorporated herein in their entireties by reference). In particular, amino acid substitutions at Arg⁸, Arg⁹, Tyr¹¹, and Ile¹² have yielded analogues that are centrally active after peripheral administration.

The hexapeptide N-Me-L-Arg-L-Lys-L-Pro-L-Trp-t-Leu-L-Leu (SEQ ID NO:2) was the first NT(8-13) analogue that elicited behavioral effects after peripheral administration. However, the various modifications incorporated in this peptide resulted in a 700-fold loss of binding affinity at NTR₁. In addition, this analogue was not able to elicit central activity after oral administration.

More recently, an NT(8-13) analogue, named NT69L, with sequence N-Me-L-Arg-L-Lys-L-Pro-L-neoTyr-t-Leu-L-Leu (SEQ ID NO:3), was shown to maintain nanomolar binding affinity at NTR₁ (K_{d}=1.55 nM) (Taylor-McMahon et al., 2000, Reg Pept. 93:125-136) and exhibit a pronounced hypothermic effect after a 1 mg/kg injection (−5.3° C. at 90 min post-injection; Taylor-McMahon et al., 2000, Eur. J. Pharmacol. 390:107-111). NT69L also attenuated hyperactivity induced by both cocaine and d-amphetamine. However, chronic administration of NT69L led to tolerance to its hypothermic effect and suppression of its d-amphetamine induced hyperactivity. As with the hexapeptide N-Me-L-Arg-L-Lys-L-Pro-L-Trp-t-Leu-L-Leu (SEQ ID NO:2), NT69L produced only a slight hypothermic response after oral administration.

The N-terminal α-methyl, α-desamino homolysyl and ornithyl analogues of NT(8-13) were synthesized and screened for activity in numerous behavioral assays predictive of antipsychotic potential (PCT Application No, WO 2006/009902, incorporated herein by reference in its entirety). These peptides induced hypothermia in a dose-dependent fashion. Administration of these peptides significantly reduced d-amphetamine induced hyperlocomotion, a measure of the therapeutic efficacy of current or potential antipsychotic drugs. Thus, the NT peptides prepared were shown to have biological activity similar to that of NT and be more selective than the naturally occurring peptide.

There is great need to develop therapeutic agents that are capable of affecting thermoregulation. Thermoregulation is essential for the maintenance of life in warm-bodied animals, because deviations from the optimum operating temperature of the body affect the rate of biochemical reactions. In humans, this optimum temperature is 36.8° C. (98.2° F.), Standard thermoregulation processes generally succeed in maintaining core body temperature within a narrow physiological limit through behavioral and autonomic mechanisms that balance heat production and loss.

Under certain circumstances, such as surgery, it is desirable to reduce the body temperature of a subject to reduce blood loss and minimize the possibility of infection. Temperature reduction may be achieved with mechanical methods, but these methods are generally restricted to peripheral application of cooling fluids or materials. A reduction in core body temperature, with a controlled and dosable medication, would facilitate procedures in the surgical room and ensure that the subject does not undergo sudden change in body temperature.

Surgery and general anesthesia impair thermoregulation by disturbing the balance between heat production and loss. In general, anesthesia, opioids and sedatives inhibit behavior and autonomic behaviors, and subjects may easily become hypothermic in cool ambient operating rooms. Conversely, hyperthermia may also occur within the perioperative period, significantly increasing the death risk in operative procedures (Nussmeier, 2005, Cardiovasc. Anesth. 32 (4):472-476). These variations in body temperature from the optimum temperature values are collectively known as temperature spiking, and may have deleterious and even fatal consequences to the subject.

Perioperative shivering is a common, yet poorly understood, surgery complication that occurs in 5-65% of subjects (Tolani & Bendo, 2007, Best Pract. & Res. Clin. Anaesth. 21(4):539-556; Buggy & Crossley, 2000, British J. Anaesth. 84:615-628). Perioperative shivering causes subject discomfort, stressful sensation of coldness, increased pain caused by muscular contractions on the operated site, increased oxygen consumption and carbon dioxide production, catecholamine release, increased cardiac output, tachycardia, decreased mixed venous oxygen saturation, hypertension and increased intracranial pressure. This increase in oxygen metabolism and demand has deleterious effect on brain activity.

Perioperative shivering is not directly connected with core hypothermia (Crossley, 1992, Anaesth. 47:193-195). Normal thermogenic shivering is initiated by the hypothalamus, based on inputs derived from temperature receptors in the skin, viscera and axis of the central nervous system. Such inputs are processed by the spinal cord and the brain before reaching the hypothalamus (Insler & Seesler, 2006, Anesth. Clin. 24:823-837). Variation in the core temperature is thus only one of the factors that may trigger perioperative shivering, and various thermal inputs influence the development of perioperative shivering (Crossley, 1995, BMJ 311:764-765). In other words, perioperative hypothermia does not necessarily cause perioperative shivering, and perioperative shivering may be observed in the absence of significant perioperative hypothermia (Horn, 1998, Anesth. 89:878-886).

Although only partially understood by scientists, perioperative shivering and temperature spiking are known to be key obstacles for successful postoperative recovery. Control of perioperative shivering reduces blood loss, duration of hospital stays and cardiac morbidity, while improving wound healing (Putzu et al., 2007, Acta Biomed. 78:163-169). Control of temperature spiking improves subject comfort, decreases cardiac morbidity, improves immune response, accelerates wound healing, and reduces chances of perioperative complications.

Mechanical procedures, such as regulating the surgical room temperature, covering the subject with surgical drapes, or controlling peripheral temperature with a forced air, radiant or resistive heating element, may interfere with the surgery itself and need to be carefully regulated to avoid unwanted oscillation in subject temperature or skin injury (Alfonsi, 2003, Minerva Anestesiol. 69:438-41).

A number of drugs, such as opioids, tramadol, alpha-2 agonists, serotonin neuromediators, corticosteroids and magnesium, have been used for the control of perioperative shivering. In terms of opioids, pethidine (a mu- and kappa-receptor agonist, alpha-2β adrenoreceptor agonist and anticholinergic agent) is thought to be effective in controlling perioperative shivering when given intravenously, while the mu-receptor agonist opioid alfentanil is thought to be less effective. The alpha-2 adrenergic agonists, such as clonidine and dexmedetomidine, may be administered one hour before the end of anesthesia to prevent shivering, without sedation or hemodynamic effects. Serotonin neuromediators, such as tramadol, ketanserin, nefopam and ondansetron, also inhibit perioperative shivering. Drugs such as methylphenidate, physostigmine or doxapram also prevent shivering by mechanisms yet to be established.

In summary, there is great interest in identifying novel molecules with biological activities similar to neurotensin and other naturally occurring endogenous peptides, especially in terms of pain management, psychosis treatment and controlled reduction in body temperature. Most of the drugs currently available to treat such conditions fall short of expectations in terms of activity, bioavailability and long-term efficacy. Perioperative shivering and temperature spiking may lead to potentially dangerous or even lethal complications, and there are limited therapy options to treat these conditions. Some of the anti-shivering drugs cause significant respiratory depression, hypotension and other side effects that negatively impact outcome. Thus, there is great need for novel medications that treat pain, treat or manage psychosis, reduce body temperature, or avoid or control perioperative shivering or temperature spiking. The present invention fulfills this need.

### SUMMARY OF THE INVENTION

The invention includes a composition comprising a compound of Formula I:

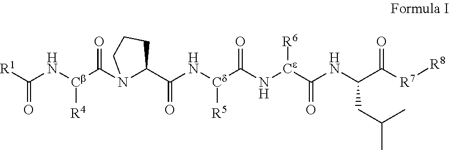

wherein:
R¹ is H, alkyl, cycloalkyl, aryl, heteroaryl, heterocyclic or C^{α}HR²R³;
R² and R⁴ are independently —(CH₂)ₘNR⁸R⁹R¹⁰, —(CH₂)ₘNR⁹C(═NR⁹)NR⁹R¹⁰, or —(CH₂)ₘ-imidazolidin-2-imin-1-yl;
R³ is —NR⁸R⁹R¹⁰, —N(R⁹)—C(═O)R⁹, —C^{φ}HR⁹R¹⁰, —C^{φ}H(R⁹)—C(═O)R¹⁰, or —C^{φ}H(C(═O)R⁹)(C(═O)R¹⁰);
R⁵ is phenyl, benzyl, —CH₂-(4-hydroxy-phenyl), —CH₂— (indol-3-yl), —CH₂-(indol-4-yl), —CH₂-(napht-1-yl), —CH₂-(napht-2-yl), —CH₂-aryl, —CH₂-(heteroaryl), napht-1-yl, or napht-2-yl;
R⁶ is methyl, ethyl, propyl, isopropyl, n-butyl, i-butyl, t-butyl, (2S)-butyl, (2R)-butyl, C₅₋₆ alkyl, cyclopropyl, cyclopropylmethyl, cyclopentyl, cyclohexyl, cyclopentylmethyl, or cyclohexylmethyl;
R⁷ is —O— or —N(R⁹)—;
R⁸, R⁹ and R¹⁰ are, independently in each instance, H, alkyl, cycloalkyl, aryl, heteroaryl, heterocyclic, or (CH₂CH₂O)ₙCH₃;
m is 1; 2, 3, 4 or 5;
n is an integer of from 1 to 20; and
C^{α}, C^{β}, C^{γ}, C^{ε} and C^{φ} are carbon atoms, and the stereochemistries at C^{α}, C^{β}, C^{γ}, C^{ε} and C^{φ} are independently either R or S; or any acceptable salt thereof.

In one embodiment, the compound is selected from the group consisting of ABS-295, ABS-296, ABS-298, ABS-334, ABS-357, ABS-358, ABS-359, ABS-363, ABS-368, ABS-398 and ABS-399. In another embodiment, the compound is ABS-363.

The invention also includes a pharmaceutical composition comprising at least one compound of Formula I;

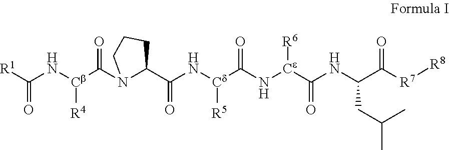

wherein:
R¹ is H, alkyl, cycloalkyl, aryl, heteroaryl, heterocyclic or C^{α}HR²R³;
R² and R⁴ are independently —(CH₂)ₘNR⁸R⁹R¹⁰, —(CH₂)ₘNR⁹C(═NR⁹)NR⁹R¹⁰, or —(CH₂)ₘ-imidazolidin-2-imin-1-yl;
R³ is —NR⁸R⁹R¹⁰, —N(R⁹)—C(═O)R⁹, —C^{φ}HR⁹R¹⁰, —C^{φ}H(R⁹)—C(═O)R¹⁰, or —C^{φ}H(C(═O)R⁹)(C(═O)R¹⁰;
R⁵ is phenyl, benzyl, —CH₂-(4-hydroxy-phenyl), —CH₂— (indol-3-yl), —CH₂— (indol-4-yl), —CH₂-(napht-1-yl), —CH₂-(napht-2-yl), —CH₂-aryl, —CH₂-(heteroaryl), napht-1-yl, or napht-2-yl;
R⁶ is methyl, ethyl, propyl, isopropyl, n-butyl, i-butyl, t-butyl, (2S)-butyl, (2R)-butyl, C₅₋₆ alkyl, cyclopropyl, cyclopropylmethyl, cyclopentyl, cyclohexyl, cyclopentylmethyl, or cyclohexylmethyl;
R⁷ is —O— or —N(R⁹)—;
R⁸, R⁹ and R¹⁰ are, independently in each instance, H, alkyl, cycloalkyl, aryl, heteroaryl, heterocyclic, or (CH₂CH₂O)ₙCH₃;
m is 1, 2, 3, 4 or 5;
n is an integer of from 1 to 20;
C^{α}, C^{β}, C^{γ}, C^{ε} and C^{φ} are carbon atoms, and the stereochemistries at C^{α}, C^{β}, C^{γ}, C^{ε} and C^{φ} are independently either Ror S;
   formulated as a pharmaceutically acceptable salt, hydrate, pro-drug or solvate thereof.

In another embodiment, the at least one compound of Formula I is selected from the group consisting of ABS-295, ABS-296, ABS-298, ABS-334, ABS-357, ABS-358, ABS-359, ABS-363, ABS-368, ABS-398 and ABS-399. In another embodiment, the at least one compound of Formula I is ABS-363.

The invention also includes a method of controlling, ameliorating or preventing shivering associated with a surgical procedure in a subject in need thereof. The method comprises the step of administering to the subject a pharmaceutical composition comprising at least one compound of Formula I:

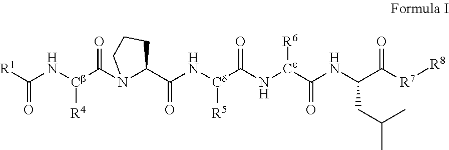

wherein:
R¹ is H, alkyl, cycloalkyl, aryl, heteroaryl, heterocyclic or C^{α}HR²R³;
R² and R⁴ are independently —(CH₂)ₘNR⁸R⁹R¹⁰, —(CH₂)ₘNR⁹C(═NR⁹)NR⁹R¹⁰, or —(CH₂)ₘ-imidazolidin-2-imin-1-yl;
R³ is —NR⁸R⁹R¹⁰, —N(R⁹)—C(═O)R⁹, —C^{φ}HR⁹R¹⁰, —C^{φ}H(R⁹)—C(═O)R¹⁰, or —C^{φ}H(C(═O)R⁹)(C(═O)R¹⁰);
R⁵ is phenyl, benzyl, —CH₂-(4-hydroxy-phenyl), —CH₂— (indol-3-yl), —CH₂— (indol-4-yl), —CH₂-(napht-1-yl), —CH₂-(napht-2-yl), —CH₂-aryl, —CH₂-(heteroaryl), napht-1-yl, or napht-2-yl;
R⁶ is methyl, ethyl, propyl, isopropyl, n-butyl, i-butyl, t-butyl, (2S)-butyl, (2R)-butyl, C₅₋₆ alkyl, cyclopropyl, cyclopropylmethyl, cyclopentyl, cyclohexyl, cyclopentylmethyl, or cyclohexylmethyl;
R⁷ is —O— or —N(R⁹)—;
R⁸, R⁹ and R¹⁰ are, independently in each instance, H, alkyl, cycloalkyl, aryl, heteroaryl, heterocyclic, or (CH₂CH₂O)ₙCHₐ;
m is 1, 2, 3, 4 or 5;
n is an integer of from 1 to 20;
C^{α}, C^{β}, C^{γ}, C^{ε} and C^{φ} are carbon atoms, and the stereochemistries at C^{α}, C^{β}, C^{γ}, C^{ε} and are independently either R or S; formulated in a pharmaceutically acceptable salt, hydrate, pro-drug or solvate thereof.

In one embodiment, the at least one compound is selected from the group consisting of ABS-295, ABS-296, ABS-298, ABS-334, ABS-357, ABS-358, ABS-359, ABS-363, ABS-368, ABS-398 and ABS-399. In another embodiment, the at least one compound is ABS-363.

In one embodiment, the method further comprises the step of administering one or more additional agents useful for treating shivering. The one or more additional agents are selected from the group consisting of an opioid, an alpha-2 agonist, a serotonin neuromediator, methylphenidate, physostigmine and doxapram. In one embodiment, the administering to the subject takes place before the surgical procedure. In another embodiment, the administering to the subject takes place during the surgical procedure. In yet another embodiment, the administering to the subject takes place after the surgical procedure.

In one embodiment, the subject is feline, canine or human. In another embodiment, the subject is human.

The invention also includes a method of controlling, ameliorating or preventing temperature spiking associated with a surgical procedure in a subject in need thereof. The method comprises the step of administering to the subject a pharmaceutical composition comprising at least one compound of Formula I:

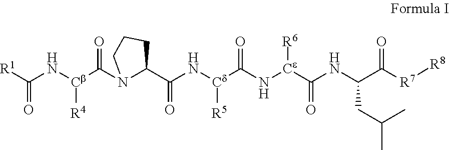

wherein:
R¹ is H, alkyl, cycloalkyl, aryl, heteroaryl, heterocyclic or C^{α}HR²R³;
R² and R⁴ are independently —(CH₂)ₘNR⁸R⁹R¹⁰, —(CH₂)ₘNR⁹C(═NR⁹)NR⁹R¹⁰, or —(CH₂)ₘ-imidazolidin-2-imin-1-yl;

R³ is —NR⁸R⁹R¹⁰, —N(—C₁₋₁₃)R⁹R¹⁰, —N(R⁹)—C(═O)R⁹, —C^{φ}HR⁹R¹⁰, —C^{φ}H(R⁹)—C(═O)R¹⁰, or —C^{φ}H(C(═O)R⁹)(C(═O)R¹⁰);
R⁵ is phenyl, benzyl, —CH₂-(4-hydroxy-phenyl), —CH₂— (indol-3-yl), —C₁₋₁₂-(indol-4-yl), —CH₂-(napht-1-yl), —CH₂-(napht-2-yl), —CH₂-aryl, —CH₂-(heteroaryl), napht-1-yl, or napht-2-yl;
R⁶ is methyl, ethyl, propyl, isopropyl, n-butyl, 1-butyl, t-butyl, (2S)-butyl, (2R)-butyl, C₅₋₆ alkyl, cyclopropyl, cyclopropylmethyl, cyclopentyl, cyclohexyl, cyclopentylmethyl, or cyclohexylmethyl;
R⁷ is —O— or —N(R⁹)—;

R⁸, R⁹ and R¹⁰ are, independently in each instance, H, alkyl, cycloalkyl, aryl, heteroaryl, heterocyclic, or (CH₂CH₂O)ₙCH₃;
m is 1, 2, 3, 4 or 5;
n is an integer of from 1 to 20;
C^{α}, C^{β}, C^{γ}, C^{ε} and C^{φ} are carbon atoms, and the stereochemistries at C^{α}, C^{β}, C^{γ}, C^{ε} and C^{φ} are independently either R or S; formulated in a pharmaceutically acceptable salt, hydrate, pro-drug or solvate thereof.

In one embodiment, the at least one compound is selected from the group consisting of ABS-295, ABS-296, ABS-298, ABS-334, ABS-357, ABS-358, ABS-359, ABS-363, ABS-368, ABS-398 and ABS-399. In another embodiment, the at least one compound is ABS-363.

In one embodiment, the administering to the subject takes place before the surgical procedure. In another embodiment, the administering to the subject takes place during the surgical procedure. In yet another embodiment, the administering to the subject takes place after the surgical procedure.

In one embodiment, the subject is feline, canine or human. In another embodiment, the subject is human.

The invention includes a method of controlling, ameliorating or preventing shivering associated with a surgical procedure in a subject in need thereof. The method comprises the step of administering to the subject a pharmaceutical composition comprising at least one compound of Formula II:

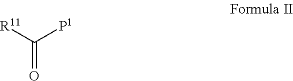

wherein
P¹ is a known or novel peptide;
R¹¹ consists of one of Formulas IIa, IIb, IIc and IId, wherein the N-terminus amine group of P′ is covalently coupled to the R¹¹C(O)— group through a peptide bond, wherein:
(a) Formula IIa is:

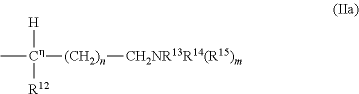

wherein
   n is 0, 1, 3, 4, or 5;
   m is zero or an integer of 1;
   R¹² is hydrogen, a straight or branched chain alkyl group of C₁-C₁₀, a cycloalkyl group of C₃-C₆, an aromatic group of C₆-C₁₈ or a corresponding substituted aromatic group with one or two substituents selected from halogen, alkyloxy, carboxy, amide or alkyl in any combination, or a heteroaromatic group of C₄-C₁a and one or two heteroatoms selected from oxygen, sulfur and nitrogen in any combination or a corresponding substituted heteroaromatic group with one or two substituents selected from halogen, alkyloxy, carboxy, amide or alkyl in any combination;
   R¹³, R¹⁴ and R¹⁵ are, independently, hydrogen or branched or straight chain alkyl, alkenyl or alkynyl of C₁-C₁₀, an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination and with the proviso that a maximum of two of R¹³, R¹⁴ and R¹⁵ may be selected to be the aromatic, substituted aromatic, heteroaromatic or substituted heteroaromatic group; and
   C^{η} is a carbon atom and the stereochemistry at C is either R or S;
(b) Formula IIb is:

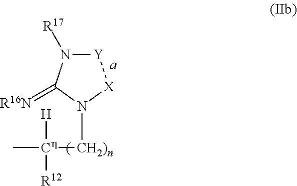

wherein
   n is an integer of from 0 to 6;
   when dashed line a is not present, X and Y are, independently, hydrogen or lower branched or straight chain alkyl, alkenyl or alkynyl of C₁-C₆;
   when dashed line a is present, X—Y is (CH₂)_{z}, wherein z is an integer of from 1 to 8;
   R¹² is hydrogen, a straight or branched chain alkyl group of C₁-C₁₀, a cycloalkyl group of C₃-C₆, an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from
   halogen, alkyloxy, carboxy, amide or alkyl in any combination;
      R¹⁶ and R¹⁷ are independently hydrogen, lower branched or straight chain alkyl of C₁-C₁₀, lower branched or straight chain alkenyl of C₁-C₁₀, lower branched or straight chain alkynyl of C₁-C₁₀, an aromatic group of C₆-C₁₈ or a corresponding substituted aromatic group with one or two substituents selected from halogen, alkyloxy, carboxy, amide or alkyl in any combination, a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination;
      C^{η} is a carbon atom and the stereochemistry at C is either R or S;
(c) Formula IIc is:

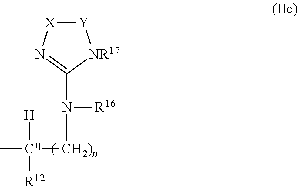

wherein
   n is an integer of from 0 to 5;
   X—Y is (CH₂)_{z}, wherein z is an integer of from 0 to 6;
   R¹² is hydrogen, a straight or branched chain alkyl group of C₁-C₁₀, a cycloalkyl group of C₃-C₆, an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination;
   R¹⁶ and R¹⁷ are independently hydrogen, lower branched or straight chain alkyl of C₁-C₁₀, lower branched or straight chain alkenyl of C₁-C₁₀, lower branched or straight chain alkynyl of C₁-C₁₀, an aromatic group of C₆-C₁₈ or a corresponding substituted aromatic group with one or two substituents selected from halogen, alkyloxy, carboxy, amide or alkyl in any combination, a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination;
   C_{η} is a carbon atom and the stereochemistry at C is either R or S;
(d) Formula IId is:

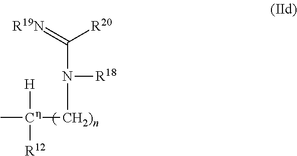

wherein
   n is an integer of from 0 to 5;
   R¹² is hydrogen, a straight or branched chain alkyl group of C₁-C₁₀, a cycloalkyl group of C₃-C₆, an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination;
   R¹⁸, R¹⁹, and R²⁰ are, independently, hydrogen or lower branched or straight chain alkyl, a cycloalkyl group of C₃-C₆, alkenyl or alkynyl of C₁-C₁₀, an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination, with the proviso that a maximum of two of R¹⁸, R¹⁹, and R²⁰ may be selected to be the aromatic, substituted aromatic, heteroaromatic or substituted heteroaromatic group; and
   C^{η} is a carbon atom and the stereochemistry at C is either R or S; and
      formulated as a pharmaceutically acceptable salt, hydrate, pro-drug or solvate thereof.

In one embodiment, P¹ is H₂N-L-Arg-L-Pro-L-Tyr-L-Ile-L-Leu-COOH (SEQ ID NO:44). In another embodiment, P′ is H₂N-L-Arg-L-Pro-L-Tyr-L-tLeu-L-Leu-COOH (SEQ ID NO:45). In yet another embodiment, the at least one compound is selected from the group consisting of ABS-201, ABS-211, ABS-212, ABS-214, ABS-220, ABS-230, ABS-234, ABS-244, ABS-262, ABS-263 and ABS-264. In yet another embodiment, P¹ is H₂N-L-Lys-L-Pro-L-Tyr-L-tLeu-L-Leu-COOH (SEQ ID NO:46). In yet another embodiment, P¹ is H₂N-L-Lys-L-Pro-L-Trp-L-tLeu-L-Leu-COOH (SEQ ID NO:47). In yet another embodiment, P′ is H₂N-L-Arg-L-Pro-L-Trp-L-tLeu-L-Leu-COOH (SEQ ID NO:48).

In one embodiment, the method further comprises the step of administering one or more additional agents useful for treating shivering. The one or more additional agents are selected from the group consisting of an opioid, an alpha-2 agonist, a serotonin neuromediator, methylphenidate, physostigmine and doxapram.

In one embodiment, the administering to the subject takes place before the surgical procedure. In another embodiment, the administering to the subject takes place during the surgical procedure. In yet another embodiment, the administering to the subject takes place after the surgical procedure.

In one embodiment, the subject is canine, feline or human. In another embodiment, the subject is human.

The invention also includes a method of controlling, ameliorating or preventing shivering associated with a surgical procedure in a subject in need thereof.

The method comprises the step of administering to the subject a pharmaceutical composition comprising at least one neo-Trp-containing peptide selected from the group consisting of PP-1 (SEQ ID NO:26), PP-2 (SEQ ID NO:27), PP-3 (SEQ ID NO:28), PP-4 (SEQ ID NO:29), PP-5 (SEQ ID NO:30), PP-6 (SEQ ID NO:31), PP-7 (SEQ ID NO:32), PP-8 (SEQ ID NO:33), PP-9 (SEQ ID NO:34), PP-10 (SEQ ID NO:35), PP-11 (SEQ ID NO:36), PP-12 (SEQ ID NO:37), PP-13 (SEQ ID NO:38), PP-14 (SEQ ID NO:39), PP-15 (SEQ ID NO:40), PP-16 (SEQ ID NO:41), PP-17 (SEQ ID NO:42) and PP-18 (SEQ ID NO:43), wherein the at least one neo-Trp-containing peptide is formulated as a pharmaceutically acceptable salt, hydrate, pro-drug or solvate thereof.

In one embodiment, the method further comprises the step of administering one or more additional agents useful for treating shivering. The one or more additional agents are selected from the group consisting of an opioid, an alpha-2 agonist, a serotonin neuromediator, methylphenidate, physostigmine and doxapram.

In one embodiment, the administering to the subject takes place before the surgical procedure. In another embodiment, the administering to the subject takes place during the surgical procedure. In yet another embodiment, the administering to the subject takes place after the surgical procedure.

In one embodiment, the subject is canine, feline or human. In another embodiment, the subject is human.

The invention also includes a method of controlling, ameliorating or preventing pain in a subject in need thereof. The method comprises administering to the subject a pharmaceutical composition comprising at least one compound of Formula I:

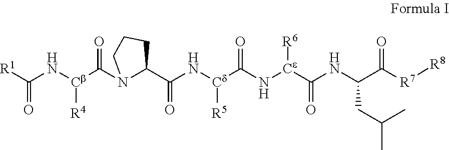

wherein:
R¹ is H, alkyl, cycloalkyl, aryl, heteroaryl, heterocyclic or C^{α}HR²R³;
R² and R⁴ are independently —(CH₂)ₘNR⁸R⁹R¹⁰, —(CH₂)ₘNR⁹C(═NR⁹)NR⁹R¹⁰, or —(CH₂)ₘ-imidazolidin-2-imin-1-yl;
R³ is —NR⁸R⁹R¹⁰, —N(R⁹)—C(═O)R⁹, —C^{φ}HR⁹R¹⁰, —C^{φ}H(R⁹)—C(═O)R¹⁰, or —C^{φ}H(C(═O)R⁹)(C(═O)R¹⁰);
R⁵ is phenyl, benzyl, —CH₂-(4-hydroxy-phenyl), —CH₂— (indol-3-yl), —C₁₋₁₂-(indol-4-yl), —CH₂-(napht-1-yl), —CH₂-(napht-2-yl), —CH₂-aryl, —CH₂-(heteroaryl), napht-1-yl, or napht-2-yl;
R⁶ is methyl, ethyl, propyl, isopropyl, n-butyl, i-butyl, t-butyl, (2S)-butyl, (2R)-butyl, C₅₋₆ alkyl, cyclopropyl, cyclopropylmethyl, cyclopentyl, cyclohexyl, cyclopentylmethyl, or cyclohexylmethyl;
R⁷ is —O— or —N(R⁹)—;
R⁸, R⁹ and R¹⁰ are, independently in each instance, H, alkyl, cycloalkyl, aryl, heteroaryl, heterocyclic, or (CH₂CH₂O)ₙCH₃;
m is 1, 2, 3, 4 or 5;
n is an integer of from 1 to 20;
C^{α}, C^{β}, C^{γ}, C^{ε} and C^{φ} are carbon atoms, and the stereochemistries at C^{α}, C^{β}, C^{γ}, C^{ε} and C^{φ} are independently either R or S; formulated in a pharmaceutically acceptable salt, hydrate, pro-drug or solvate thereof.

In one embodiment, the at least one compound is selected from the group consisting of ABS-295, ABS-296, ABS-298, ABS-334, ABS-357, ABS-358, ABS-359, ABS-363, ABS-368, ABS-398 and ABS-399. In another embodiment, the at least one compound is ABS-363.

In one embodiment, the subject is canine, feline or human. In another embodiment, the subject is human.

The invention includes a method of controlling, ameliorating or preventing psychosis in a subject in need thereof. The method comprises administering to the subject a pharmaceutical composition comprising at least one compound of Formula

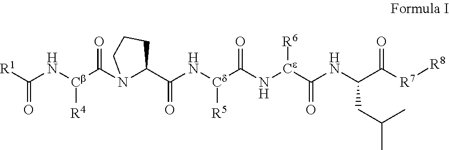

wherein:
R¹ is H, alkyl, cycloalkyl, aryl, heteroaryl, heterocyclic or C^{α}HR²R³;
R² and R⁴ are independently —(CH₂)ₘNR⁸R⁹R¹⁰, —(CH₂)ₘNR⁹C(═NR⁹)NR⁹R¹⁰, or —(CH₂)ₘ-imidazolidin-2-imin-1-yl;
R³ is —NR⁸R⁹R¹⁰, —N(R⁹)—C(═O)R⁹, —C^{φ}HR⁹R¹⁰, —C^{φ}H(R⁹)—C(═O)R¹⁰, or —C^{φ}H(C(═O)R⁹)(C(═O)R¹⁰);
R⁵ is phenyl, benzyl, —CH₂-(4-hydroxy-phenyl), —CH₂— (indol-3-yl), —CH₂-(indol-4-yl), —CH₂-(napht-1-yl), —CH₂-(napht-2-yl), —CH₂-aryl, —CH₂-(heteroaryl), napht-1-yl, or napht-2-yl;
R⁶ is methyl, ethyl, propyl, isopropyl, n-butyl, i-butyl, t-butyl, (2S)-butyl, (2R)-butyl, C₅₋₆ alkyl, cyclopropyl, cyclopropylmethyl, cyclopentyl, cyclohexyl, cyclopentylmethyl, or cyclohexylmethyl;
R⁷ is —O— or —N(R⁹)—;
R⁸, R⁹ and R¹⁰ are, independently in each instance, H, alkyl, cycloalkyl, aryl, heteroaryl, heterocyclic, or (CH₂CH₂O)ₙCH₃;
m is 1, 2, 3, 4 or 5;
n is an integer of from 1 to 20;
C^{α}, C^{β}, C^{γ}, C^{ε} and C^{φ} are carbon atoms, and the stereochemistries at C^{α}, C^{β}, C^{γ}, C^{ε} and C^{φ} are independently either R or S;
   formulated in a pharmaceutically acceptable salt, hydrate, pro-drug or solvate thereof.

In one embodiment, the at least one compound is selected from the group consisting of ABS-295, ABS-296, ABS-298, ABS-334, ABS-357, ABS-358, ABS-359, ABS-363, ABS-368, ABS-398 and ABS-399. In another embodiment, the at least one compound is ABS-363.

In one embodiment, the psychosis is schizophrenia. In another embodiment, the subject is human.

The invention includes a method of lowering the body temperature of a subject in need thereof. The method comprises the step of administering to the subject a pharmaceutical composition comprising at least one compound of Formula I:

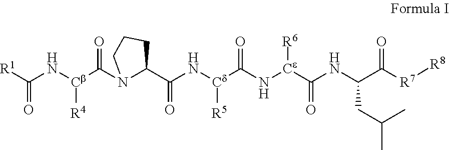

wherein:
R¹ is H, alkyl, cycloalkyl, aryl, heteroaryl, heterocyclic or C^{α}HR²R³;
R² and R⁴ are independently —(CH₂)ₘNR⁸R⁹R¹⁰, —(CH₂)ₘNR⁹C(═NR⁹)NR⁹R¹⁰, or —(CH₂)ₘ-imidazolidin-2-imin-1-yl;

R³ is —NR⁸R⁹R¹⁰, —N(R⁹)—C(═O)R⁹, —C+HR⁹R¹⁰, —C+H(R⁹)—C(═O)R¹⁰, or —C+H(C(═O)R⁹)(C(═O)R¹⁰);
R⁵ is phenyl, benzyl, —CH₂-(4-hydroxy-phenyl), —CH₂— (indol-3-yl), —CH₂— (indol-4-yl), —CH₂-(napht-1-yl), —CH₂-(napht-2-yl), —CH₂-aryl, —CH₂-(heteroaryl), napht-1-yl, or napht-2-yl;
R⁶ is methyl, ethyl, propyl, isopropyl, n-butyl, i-butyl, t-butyl, (2S)-butyl, (2R)-butyl, C₅₋₆ alkyl, cyclopropyl, cyclopropylmethyl, cyclopentyl, cyclohexyl, cyclopentylmethyl, or cyclohexylmethyl;
R⁷ is —O— or —N(R⁹)—;
R⁸, R⁹ and R¹⁰ are, independently in each instance, H, alkyl, cycloalkyl, aryl, heteroaryl, heterocyclic, or (CH₂CH₂O)ₙCH₃;
m is 1, 2, 3, 4 or 5;
n is an integer of from 1 to 20;
C^{α}, C^{β}, C^{γ}, C^{ε} and C^{φ} are carbon atoms, and the stereochemistries at C^{α}, C^{β}, C^{γ}, C^{ε} and C^{φ} are independently either R or S;
   formulated in a pharmaceutically acceptable salt, hydrate, pro-drug or solvate thereof.

In one embodiment, the at least one compound is selected from the group consisting of ABS-295, ABS-296, ABS-298, ABS-334, ABS-357, ABS-358, ABS-359, ABS-363, ABS-368, ABS-398 and ABS-399. In another embodiment, the at least one compound is ABS-363.

In one embodiment, the subject is feline, canine or human. In another embodiment, the subject is human.

### DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 illustrates the chemical structure of compounds of Formula I of the invention.

FIG. 2 illustrates the chemical structure of the compounds of Formula II of the invention.

FIG. 3 illustrates the chemical structure of compounds ABS-201 (SEQ ID NO:4), ABS-211 (SEQ ID NO:5), ABS-212 (SEQ ID NO:6), ABS-214 (SEQ ID NO:7), ABS-220 (SEQ ID NO:8), and ABS-230 (SEQ ID NO:9).

FIG. 4 illustrates the chemical structure of compounds ABS-234 (SEQ ID NO:10), ABS-244 (SEQ ID NO:11), ABS-262 (SEQ ID NO:12), ABS-263 (SEQ ID NO:13), and ABS-264 (SEQ ID NO:14).

FIG. 5 illustrates the chemical structure of compounds ABS-295 (SEQ ID NO:15), ABS-296 (SEQ ID NO:16), ABS-298 (SEQ ID NO:17), ABS-334 (SEQ ID NO:18) and ABS-357 (SEQ ID NO:19).

FIG. 6 illustrates the chemical structure of compounds ABS-358 (SEQ ID NO:20), ABS-359 (SEQ ID NO:21), ABS-363 (SEQ ID NO:22), ABS-368 (SEQ ID NO:23), ABS-398 (SEQ ID NO:24) and ABS-399 (SEQ ID NO:25).

FIG. 7 illustrates the sequence of the neo-Trp-containing peptides of the invention: PP-1 (SEQ ID NO:26), PP-2 (SEQ ID NO:27), PP-3 (SEQ ID NO:28), PP-4 (SEQ ID NO:29), PP-5 (SEQ ID NO:30), PP-6 (SEQ ID NO:31), PP-7 (SEQ ID NO:32), PP-8 (SEQ ID NO:33), PP-9 (SEQ ID NO:34), PP-10 (SEQ ID NO:35), PP-11 (SEQ ID NO:36), PP-12 (SEQ ID NO:37), PP-13 (SEQ ID NO:38), PP-14 (SEQ ID NO:39), PP-15 (SEQ ID NO:40), PP-16 (SEQ ID NO:41), PP-17 (SEQ ID NO:42) and PP-18 (SEQ ID NO:43).

FIG. 8 is a graph illustrating the time-dependent core body temperature of monkeys (in Celsius degrees) administered with ABS-212. The data set for male monkeys is presented as a broken line, and the data set for female monkeys is presented as a continuous line.

FIG. 9 is a graph illustrating the core body temperature of rats administered with ABS-212 (continuous line) and with saline control (broken line).

FIG. 10 is a graph illustrating the temperature changes in male Sprague-Dawley rats induced by i.v. administration of ABS-363 at different doses.

FIG. 11 is a graph illustrating the temperature changes in two monkeys, one male and one female, when ABS-363 (0.1 mg/kg dose) was administered.

FIG. 12, comprising FIGS. 12a-12b, is a set of graphs illustrating the decrease in body temperature in rats caused by i.p. dosing of ABS-201 (structure shown).

FIG. 12a illustrates the change in body temperature as a function of time for each dosing. FIG. 12b illustrates shows the correlation between the maximal change in body temperature and the dosing used (in log scale).

FIG. 13 is a graph illustrating the mean core and chamber temperature recordings for a rat treated with ABS-363 (0.1 mg/kg; 1 mL/kg) and saline (control).

FIG. 14 is a series of graphs illustrating EMG amplitudes (in V) registered in a shiver chamber for a rat treated with ABS-363 (0.1 mg/kg; 1 mL/kg) and saline (control).

### DETAILED DESCRIPTION OF INVENTION

As described herein, the invention relates to the discovery that shivering associated with surgical intervention in a subject may be controlled, ameliorated or avoided by administration of the compounds of the invention to the subject.

The invention also relates to the discovery that temperature spiking associated with surgical intervention in a subject may be controlled, ameliorated or avoided by administration of the compounds of the invention to the subject.

The invention also relates to the discovery that psychosis in a subject may be treated, controlled, ameliorated or avoided by administration of compounds of the invention to the subject.

The invention also relates to the discovery that pain in a subject may be treated, controlled, ameliorated or avoided by administration of compounds of the invention to the subject.

The invention also relates to the discovery that administration of the compounds of the invention to a subject may be used to lower the body temperature of the subject.

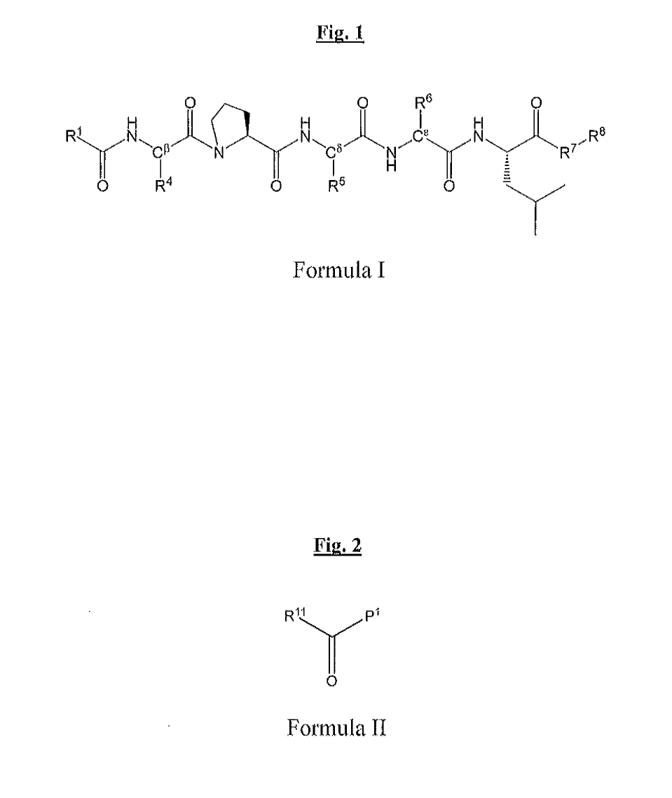

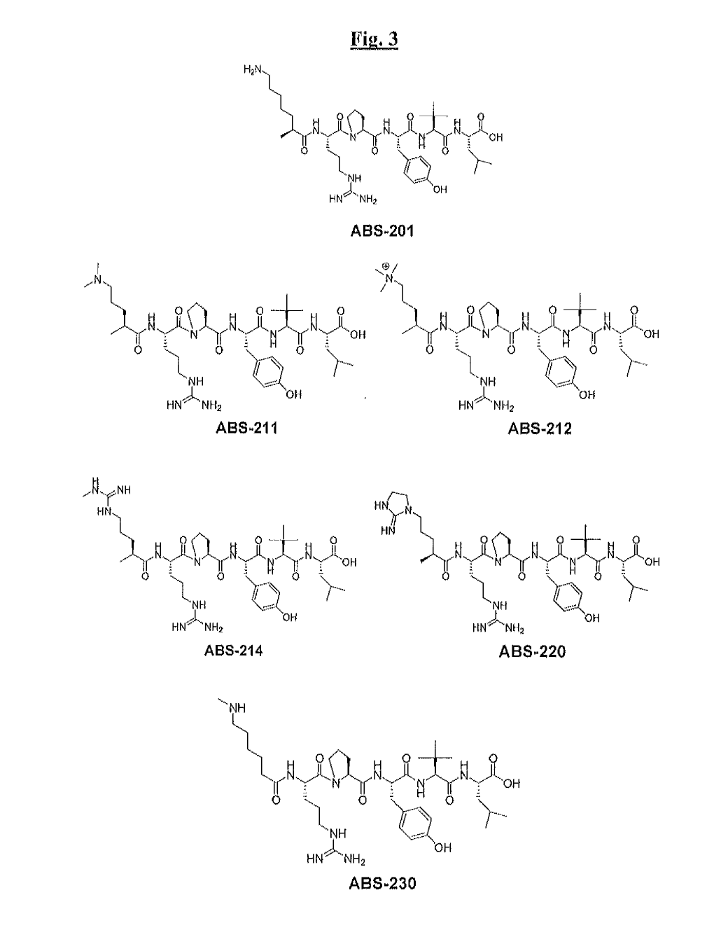

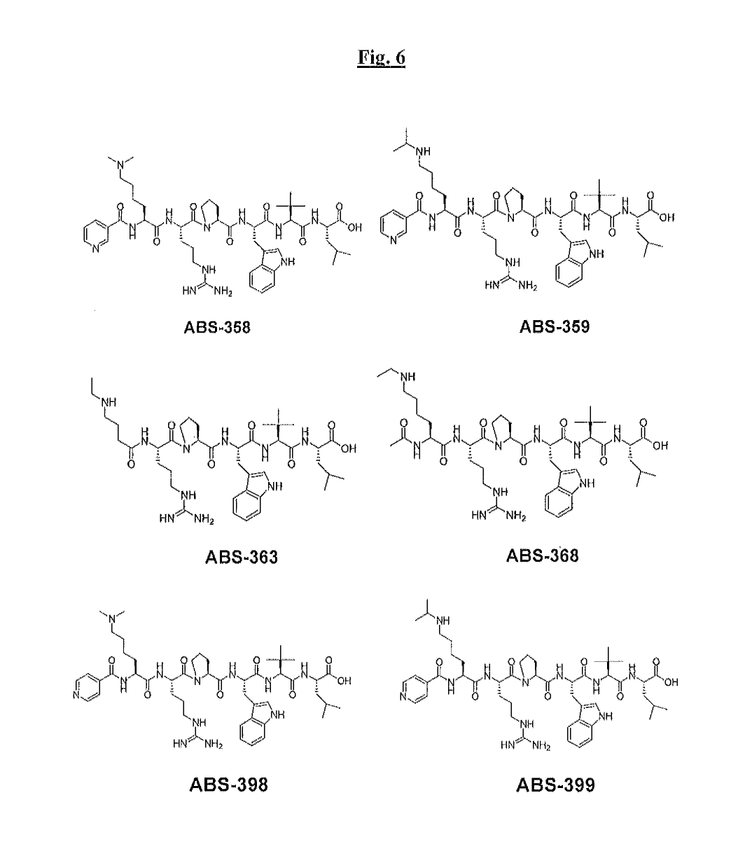

The invention includes compounds encompassed by Formula I, as shown in FIG. 1. In one embodiment, the compounds are selected from the group consisting of ABS-295 (SEQ ID NO:15), ABS-296 (SEQ ID NO:16), ABS-298 (SEQ ID NO:17), ABS-334 (SEQ ID NO:18), ABS-357 (SEQ ID NO:19), ABS-358 (SEQ ID NO:20), ABS-359 (SEQ ID NO:21), ABS-363 (SEQ ID NO:22), ABS-368 (SEQ ID NO:23), ABS-398 (SEQ ID NO:24) and ABS-399 (SEQ ID NO:25). In another embodiment, the compound is ABS-363. The chemical structures for these compounds are shown in FIGS. 5 and 6.

The invention further includes a pharmaceutical composition comprising at least one compound of Formula I. In one embodiment, the pharmaceutical composition comprises at least one compound selected from the group consisting of ABS-295, ABS-296, ABS-298, ABS-334, ABS-357, ABS-358, ABS-359, ABS-363, ABS-368, ABS-398 and ABS-399. In another embodiment, the pharmaceutical composition comprises ABS-363.

The invention further includes a method of treating, controlling, ameliorating or avoiding psychosis in a subject in need thereof. The method comprises administering to the subject an effective amount of a pharmaceutical composition comprising at least one compound of Formula I. In an embodiment, the pharmaceutical composition comprises at least one compound selected from the group consisting of ABS-295, ABS-296, ABS-298, ABS-334, ABS-357, ABS-358, ABS-359, ABS-363, ABS-368, ABS-398 and ABS-399. In another embodiment, the pharmaceutical composition comprises ABS-363. In yet another embodiment, the psychosis is schizophrenia.

The invention further includes a method of treating, controlling, ameliorating or avoiding pain in a subject in need thereof. The method comprises administering to the subject an effective amount of a pharmaceutical composition comprising at least one compound of Formula I. In one embodiment, the pharmaceutical composition comprises at least one compound selected from the group consisting of ABS-295, ABS-296, ABS-298, ABS-334, ABS-357, ABS-358, ABS-359, ABS-363, ABS-368, ABS-398 and ABS-399. In another embodiment, the pharmaceutical composition comprises ABS-363.

The invention further includes a method of lowering the body temperature in a subject in need thereof. The method comprises administering to the subject an effective amount of a pharmaceutical composition comprising at least one compound of Formula I. In one embodiment, the pharmaceutical composition comprises at least one compound selected from the group consisting of ABS-295, ABS-296, ABS-298, ABS-334, ABS-357, ABS-358, ABS-359, ABS-363, ABS-368, ABS-398 and ABS-399. In another embodiment, the pharmaceutical composition comprises ABS-363.

The invention further includes a method of controlling, ameliorating or preventing shivering associated with surgical intervention in a subject in need thereof. The method comprises administering to the subject an effective amount of a pharmaceutical composition comprising at least one compound of Formula I. In one embodiment, the pharmaceutical composition comprises at least one compound selected from the group consisting of ABS-295, ABS-296, ABS-298, ABS-334, ABS-357, ABS-358, ABS-359, ABS-363, ABS-368, ABS-398 and ABS-399. In another embodiment, the pharmaceutical composition comprises ABS-363.

The invention further includes a method of controlling, ameliorating or preventing temperature spiking associated with surgical intervention in a subject in need thereof. The method comprises administering to the subject an effective amount of a pharmaceutical composition comprising at least one compound of Formula I. In one embodiment, the pharmaceutical composition comprises at least one compound selected from the group consisting of ABS-295, ABS-296, ABS-298, ABS-334, ABS-357, ABS-358, ABS-359, ABS-363, ABS-368, ABS-398 and ABS-399. In another embodiment, the pharmaceutical composition comprises ABS-363.

The invention further includes a method of controlling, ameliorating or preventing shivering associated with surgical intervention in a subject in need thereof. The method comprises administering to the subject an effective amount of a pharmaceutical composition comprising at least one compound of Formula II.

In one embodiment, in Formula II, P¹ is H₂N-L-Arg-L-Pro-L-Tyr-L-Ile-L-Leu-COOH (SEQ ID NO:44).

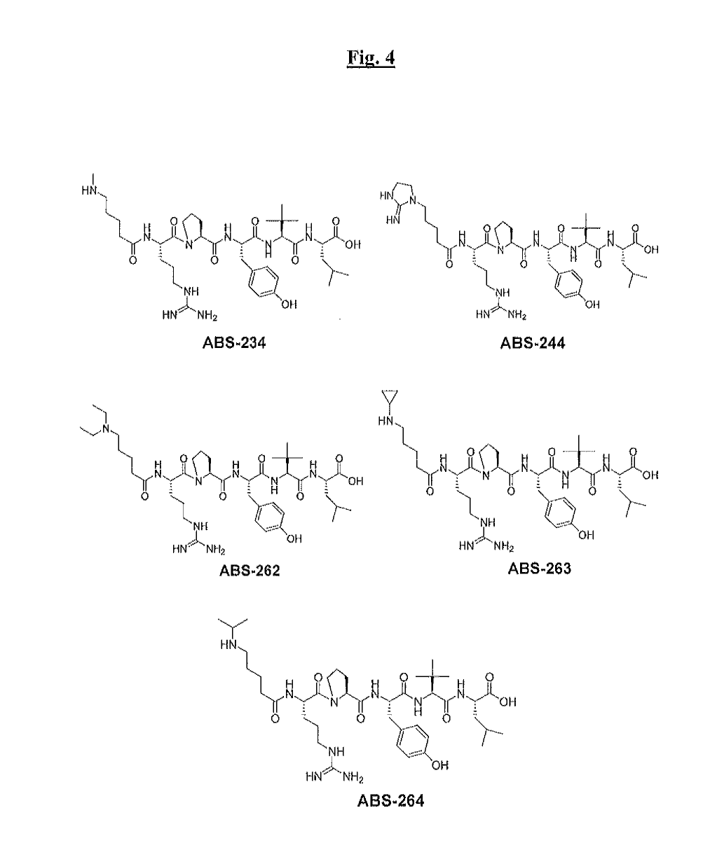

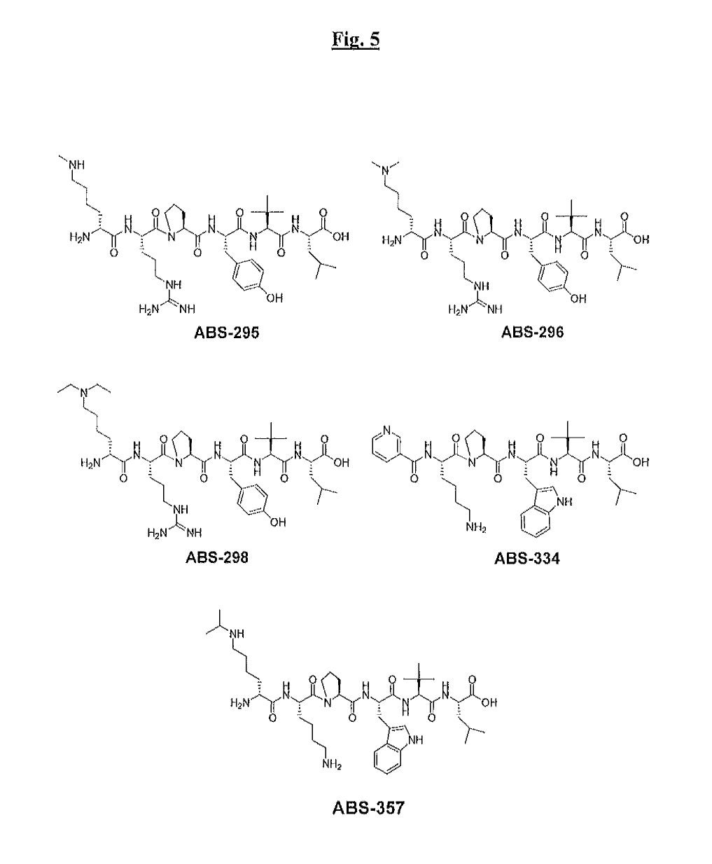

In another embodiment, in Formula II, P′ is H₂N-L-Arg-L-Pro-L-Tyr-tLeu-L-Leu-COOH (SEQ ID NO:45). In a preferred aspect, the at least one compound of Formula II is selected from the group consisting of ABS-201 (SEQ ID NO:4), ABS-211 (SEQ ID NO:5), ABS-212 (SEQ ID NO:6), ABS-214 (SEQ ID NO:7), ABS-220 (SEQ ID NO:8), ABS-230 (SEQ ID NO:9), ABS-234 (SEQ ID NO:10), ABS-244 (SEQ ID NO:11), ABS-262 (SEQ ID NO:12), ABS-263 (SEQ ID NO:13), and ABS-264 (SEQ ID NO:14). The chemical structures of these compounds are shown in FIGS. 3 and 4.

In another embodiment, in Formula II, P′ is H₂N-L-Lys-L-Pro-L-Tyr-L-tLeu-L-Leu-COOH (SEQ ID NO:46).

In another embodiment, in Formula II, P¹ is H₂N-L-Lys-L-Pro-L-Trp-L-tLeu-L-Leu-COOH (SEQ ID NO:47).

In another embodiment, in Formula II, P′ is H₂N-L-Arg-L-Pro-L-Trp-L-tLeu-L-Leu-COOH (SEQ ID NO:48).

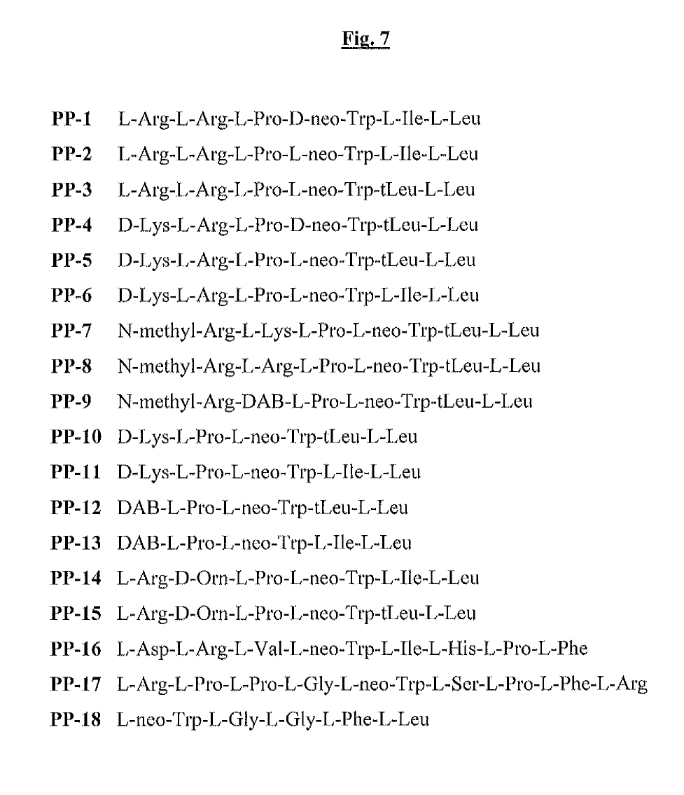

The invention further provides a method of controlling, ameliorating or preventing shivering associated with surgical intervention in a subject in need thereof. The method comprises administering to the subject an effective amount of a pharmaceutical composition comprising at least one neo-Trp-containing peptide. In one embodiment, the at least one neo-Trp-containing peptide is selected from the group consisting of PP-1 (SEQ ID NO:26), PP-2 (SEQ ID NO:27), PP-3 (SEQ ID NO:28), PP-4 (SEQ ID NO:29), PP-5 (SEQ ID NO:30), PP-6 (SEQ ID NO:31), PP-7 (SEQ ID NO:32), PP-8 (SEQ ID NO:33), PP-9 (SEQ ID NO:34), PP-10 (SEQ ID NO:35), PP-11 (SEQ ID NO:36), PP-12 (SEQ ID NO:37), PP-13 (SEQ ID NO:38), PP-14 (SEQ ID NO:39), PP-15 (SEQ ID NO:40), PP-16 (SEQ ID NO:41), PP-17 (SEQ ID NO:42) and PP-18 (SEQ ID NO:43). Hereinafter, the term “neo-Trp-containing peptide” refers to any of the compounds above (PP-1 through PP-18). The chemical structures for compounds PP-1 through PP-18 are shown in FIG. 7.

In one embodiment, the pharmaceutical composition comprising at least one of the compounds of the invention is formulated using one or more pharmaceutically acceptable excipients selected from the group consisting of starch, sugar, cellulose, diluent, granulating agent, lubricant, binder, disintegrating agent, wetting agent, emulsifier, coloring agent, release agent, coating agent, sweetening agent, flavoring agent, perfuming agent, preservative, antioxidant, plasticizer, gelling agent, thickener, hardener, setting agent, suspending agent, surfactant, humectant, carrier, stabilizer, and any combinations thereof.

In one embodiment, the pharmaceutical composition comprising at least one of the compounds of the invention is administered to the subject orally, nasally, rectally, intravaginally, parenterally, buccally, sublingually, intragastrically or topically. Preferably, the subject is a mammal. More preferably, the subject is feline, canine or human. Even more preferably, the subject is a human.

In one embodiment, the compounds of Formula I, compounds of Formula II or neo-Trp-containing peptides may be used in the methods of the present invention in combination with one or more mechanical treatments associated with the control of perioperative shivering.

In one embodiment, the compounds of Formula I, compounds of Formula II or neo-Trp-containing peptides may be used within the methods of the present invention in combination with at least one additional agent useful for treating perioperative shivering. The additional agent is known to treat, prevent, or reduce the symptoms of perioperative shivering. In one embodiment, the at least one additional agent is selected from the group consisting of opioids, alpha-2 agonists, serotonin neuromediators, methylphenidate, physostigmine and doxapram. In one aspect, the opioid comprises pethidine or alfentanil. In another aspect, the alpha-2 agonist comprises clonidine or dexmedetomidine. In yet another aspect, the serotonin neuromediator comprises tramadol, ketanserin, nefopam or ondansetron.

In one embodiment, the therapeutic effect achieved in administering the compound of the invention in combination with at least one additional agent useful for treating perioperative shivering is synergistic, wherein the overall therapeutic effect is greater than the sum of the therapeutic effects achieved by administering either a compound of the invention or the at least one additional agent alone.

In one embodiment, the compound of the invention may be administered before the surgical procedure. In another embodiment, the compound of the invention may be administered during the surgical procedure. In yet another embodiment, the compound of the invention may be administered after the surgical procedure.

### DEFINITIONS

The definitions used in this application are for illustrative purposes and do not limit the scope used in the practice of the invention.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

As used herein, the articles “a” and “an” are used to refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used.

As used herein, the term “alkyl” refers to a branched or unbranched saturated hydrocarbon group of 1 to 24 carbon atoms, such as methyl, ethyl, n-propyl, isopropyl, n-butyl, isobutyl, tert-butyl, pentyl, hexyl, heptyl, octyl, decyl and the like. Preferred alkyl groups herein contain 1 to 6 carbon atoms. Alkyl groups may be optionally substituted with one to three groups selected from halo, amino, methoxy, ethoxy, hydroxyl, methylthio, methylsulfonyl, nitro, aryl, heterocyclyl and heteroaryl.

As used herein, the term “cycloalkyl” refers to ring-containing alkyl radicals. Examples include cyclohexyl, cyclopentyl, cyclopropyl, cyclopropylmethyl and norbornyl. Cycloalkyl groups may be optionally substituted with one to three groups selected from halo, amino, methoxy, ethoxy, hydroxyl, methylthio, methylsulfonyl, nitro, aryl, heterocyclyl and heteroaryl.

As used herein, the term “aryl” employed alone or in combination with other terms means, unless otherwise stated, a carbocyclic aromatic group containing one or more rings (typically one, two or three rings). Multiple rings may be attached together in a pendent manner, such as a biphenyl, or may be fused, such as naphthalene. Examples include, but are not limited to, phenyl, anthracyl and naphthyl. Preferred are phenyl and naphthyl, most preferred is phenyl. Aryl groups may be optionally substituted with one to three groups selected from halo, amino, methoxy, ethoxy, hydroxyl, methylthio, methylsulfonyl, nitro, aryl, heterocyclyl and heteroaryl.

As used herein, the term “heterocycle”, “heterocyclyl” or “heterocyclic” by itself or as part of another substituent means, unless otherwise stated, an unsubstituted or substituted, stable, mono- or multicyclic heterocyclic ring system consisting of carbon atoms and at least one heteroatom selected from the group consisting of N, O, and S, and wherein the nitrogen and sulfur heteroatoms may be optionally oxidized, and the nitrogen atom may be optionally quaternized. The heterocycle may be attached to the compound of which it is a component, unless otherwise stated, at any heteroatom or carbon atom in the heterocycle that affords a stable structure. Heterocyclic groups may be optionally substituted with one to three groups selected from halo, amino, methoxy, ethoxy, hydroxyl, methylthio, methylsulfonyl, nitro, aryl, heterocyclyl and heteroaryl.

Non-limiting examples of non-aromatic heterocycles include monocyclic groups such as: aziridinyl, oxiranyl, thiiranyl, azetidinyl, oxetanyl, thietanyl, pyrrolidinyl, pyrrolinyl, imidazolinyl, pyrazolidinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydropyridinyl, 1,4-dihydropyridinyl, piperazinyl, morpholinyl, thiomorpholinyl, pyranyl, 2,3-dihydropyranyl, tetrahydropyranyl, 1,4-dioxanyl, 1,3-dioxanyl, homopiperazinyl, homopiperidinyl, 1,3-dioxepinyl, 4,7-dihydro-1,3-dioxepinyl and hexamethyleneoxide.

As used herein, the term “heteroaryl” or “heteroaromatic” refers to a heterocycle having aromatic character. A monocyclic heteroaryl group is preferably a 5-, 6-, or 7-membered ring, examples of which are pyrrolyl, furyl, thienyl, pyridyl, pyrimidinyl and pyrazinyl. A polycyclic heteroaryl may comprise multiple aromatic rings or may include one or more partially saturated rings. Heteroaryl groups may be optionally substituted with one to three groups selected from halo, amino, methoxy, ethoxy, hydroxyl, methylthio, methylsulfonyl, nitro, aryl, heterocyclyl and heteroaryl.

Non-limiting examples of monocyclic heteroaryl groups include, for example, six-membered monocyclic aromatic rings such as, for example, pyridyl, pyrazinyl, pyrimidinyl and pyridazinyl; and five-membered monocyclic aromatic rings such as, for example, thienyl, furyl, pyrrolyl, imidazolyl, thiazolyl, oxazolyl, pyrazolyl, isothiazolyl, 1,2,3-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Non-limiting examples of polycyclic heteroaryl groups containing a partially saturated ring include tetrahydroquinolyl and 2,3-dihydrobenzofuryl.

Non-limiting examples of polycyclic heteroaryls include indolyl, indolinyl, quinolyl, tetrahydroquinolyl, isoquinolyl, 1,2,3,4-tetrahydroisoquinolyl, cinnolinyl, quinoxalinyl, quinazolinyl, phthalazinyl, 1,8-naphthyridinyl, 1,4-benzodioxanyl, chromene-2-one-yl (coumarinyl), dihydrocoumarin, chromene-4-one-yl, benzofuryl, 1,5-naphthyridinyl, 2,3-dihydrobenzofuryl, 1,2-benzisoxazolyl, benzothienyl, benzoxazolyl, benzothiazolyl, purinyl, benzimidazolyl, benzotriazolyl, thioxanthinyl, benzazepinyl, benzodiazepinyl, carbazolyl, carbolinyl, acridinyl, pynolizidinyl and quinolizidinyl.

As used herein, the terms “peptide,” “polypeptide,” or “protein” are used interchangeably, and refer to a compound comprised of amino acid residues covalently linked by peptide bonds. A protein or peptide must contain at least two amino acids, and no limitation is placed on the maximum number of amino acids that may comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs and fusion proteins, among others. The polypeptides include natural peptides, recombinant peptides, synthetic peptides or a combination thereof. A peptide that is not cyclic will have a N-terminal and a C-terminal. The N-terminal will have an amino group, which may be free (i.e., as a NH₂ group) or appropriately protected (for example, with a BOC or a Fmoc group). The C-terminal will have a carboxylic group, which may be free (i.e., as a COOH group) or appropriately protected (for example, as a benzyl or a methyl ester). A cyclic peptide does not have free N- or C-terminal, since they are covalently bonded through an amide bond to form the cyclic structure. Amino acids may be represented by their full names (for example, leucine), 3-letter abbreviations (for example, Leu) and 1-letter abbreviations (for example, L). The structure of amino acids and their abbreviations may be found in the chemical literature, such as in Stryer, “Biochemistry”, 3^{rd} Ed., W. H. Freeman and Co., New York, 1988. tLeu represents tert-leucine. neo-Trp represents 2-amino-3-(1H-indol-4-yl)-propanoic acid. DAB is 2,4-diaminobutyric acid. Orn is ornithine. N-Me-Arg or N-methyl-Arg is 5-guanidino-2-(methylamino)pentanoic acid.

As used herein, “neo-Trp-containing peptide” refers to any of the following peptides, or any pharmaceutically acceptable salt thereof: PP-1, PP-2, PP-3, PP-4, PP-5, PP-6, PP-7, PP-8, PP-9, PP-10, PP-11, PP-12, PP-13, PP-14, PP-15, PP-16, PP-17 or PP-18, and combinations thereof. This group of compounds is also referred to as “PP-1 through PP-18”, The structures of PP-1 through PP-18 are shown in FIG. 7.

As used herein, PP-1 is L-Arg-L-Arg-L-Pro-D-neo-Trp-L-Ile-L-Leu. As used herein, PP-2 is L-Arg-L-Arg-L-Pro-L-neo-Trp-L-Ile-L-Leu. As used herein, PP-3 is L-Arg-L-Arg-L-Pro-L-neo-Trp-tLeu-L-Leu. As used herein, PP-4 is D-Lys-L-Arg-L-Pro-D-neo-Trp-tLeu-L-Leu. As used herein, PP-5 is D-Lys-L-Arg-L-Pro-L-neo-Trp-tLeu-L-Leu. As used herein, PP-6 is D-Lys-L-Arg-L-Pro-L-neo-Trp-L-Ile-L-Leu. As used herein, PP-7 is N-Me-Arg-L-Lys-L-Pro-L-neo-Trp-tLeu-L-Leu. As used herein, PP-8 is N-Me-Arg-L-Arg-L-Pro-L-neo-Trp-tLeu-L-Leu. As used herein, PP-9 is N-Me-Arg-DAB-L-Pro-L-neo-Trp-tLeu-L-Leu. As used herein, PP-10 is D-Lys-L-Pro-L-neo-Trp-tLeu-L-Leu. As used herein, PP-11 is D-Lys-L-Pro-L-neo-Trp-L-Ile-L-Leu. As used herein, PP-12 is DAB-L-Pro-L-neo-Trp-tLeu-L-Leu. As used herein, PP-13 is DAB-L-Pro-L-neo-Trp-L-Ile-L-Leu. As used herein, PP-14 is L-Arg-D-Orn-L-Pro-L-neo-Trp-L-Ile-L-Leu. As used herein, PP-15 is L-Arg-D-Orn-L-Pro-L-neo-Trp-tLeu-L-Leu. As used herein, PP-16 is L-Asp-L-Arg-L-Val-L-neo-Trp-L-Ile-L-His-L-Pro-L-Phe. As used herein, PP-17 is L-Arg-L-Pro-L-Pro-L-Gly-L-neo-Trp-L-Ser-L-Pro-L-Phe-L-Arg. As used herein, PP-18 is L-neo-Trp-L-Gly-L-Gly-L-Phe-L-Leu.

As used herein with respect to the compounds of Formula I, compounds of Formula II or neo-Trp-containing peptides, the term “biologically active” means that the compound elicits a biological response in a subject that may be monitored and characterized in comparison with an untreated subject. One possible biological response within the invention relates to the ability of the compound to avoid, reduce or treat pain in a subject. Another possible biological response within the invention relates to the ability of the compound to treat psychosis in a subject. Another possible biological response within the invention relates to the ability of the compound to avoid, reduce or treat shivering in a subject. One preferred biological response within the invention relates to the ability of the compound to induce change in body temperature in a subject. The compound may be administered to the subject orally, nasally, rectally, intravaginally, parenterally, buccally, sublingually, intragastrically or topically. The subject and its body temperature are monitored as a function of time, and the observation of a measurable and dose-dependent change in body temperature is evidence that the compound displays biological activity. This preferred biological response does not limit or restrict the disclosures or embodiments of the invention in any way.

As used herein, the term “treating” means ameliorating the effects of, or delaying, halting or reversing the progress of a disease or disorder. The word encompasses reducing the severity of a symptom of a disease or disorder and/or the frequency of a symptom of a disease or disorder.

As used herein, the term “medical intervention” means a set of one or more medical procedures or treatments that are required for ameliorating the effects of, delaying, halting or reversing a disease or disorder of a subject. A medical intervention may involve surgical procedures or not, depending on the disease or disorder in question. A medical intervention may be wholly or partially performed by a medical specialist, or may be wholly or partially performed by the subject himself or herself, if capable, under the supervision of a medical specialist or according to literature or protocols provided by the medical specialist.

As used herein, the term “subject” or “patient” refers to a human or a non-human mammal. Non-human mammals include, for example, livestock and pets, such as ovine, bovine, porcine, canine, feline and murine mammals. Preferably, the subject is canine, feline or human. More preferably, the subject is human.

As used herein, the term “effective amount” or “therapeutically effective amount” refers to a non-toxic but sufficient amount of the composition used in the practice of the invention that is effective to treat perioperative shivering or temperature spiking in a subject, or to lower the body temperature of a subject, or to avoid, reduce or treat pain in a subject, or to avoid, reduce or treat psychosis in a subject. The desired treatment may be prophylactic and/or therapeutic. That result may be reduction and/or alleviation of the signs, symptoms, or causes of a disease or disorder, or any other desired alteration of a biological system. An appropriate therapeutic amount in any individual case may be determined by one of ordinary skill in the art using routine experimentation.

The term “temperature spiking” refers to uncontrolled and unexpected deviations in body temperature of a subject from the optimum operating temperature characteristic of the subject. These variations may be positive in magnitude (leading to hyperthermia) or negative in magnitude (leading to hypothermia).

The term “synergistic”, when applied to the use of at least two drugs in a therapeutic treatment, indicates that the therapeutic benefit obtained by combining the two or more drugs in a treatment is greater than the juxtaposition of the therapeutic benefit obtained when each drug is used by itself. If the first drug provides benefit “x” and the second drug provide's benefit “y”, the benefit provided by combining the two drugs has to be greater than “x+y” to characterize synergy or synergistic properties. Synergistic drugs may be administered concomitantly or sequentially, in the same formulation or different formulations.

A “prophylactic” or “preventive” treatment indicates a treatment administered to a subject who does not exhibit signs of a disease or disorder or exhibits only early signs of the disease or disorder for the purpose of decreasing the risk of developing pathology associated with the disease or disorder.

A “therapeutic” treatment indicates a treatment administered to a subject who exhibits signs of pathology of a disease or disorder for the purpose of diminishing or eliminating those signs.

As used herein, the term “pharmaceutical composition” refers to a mixture of at least one compound of the invention with at least one pharmaceutically acceptable carrier. The pharmaceutical composition facilitates administration of the compound to an organism.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, ameliorating, or preventing shivering in a subject.

“Applicator,” as the term is used herein, is used to identify any device including, but not limited to, a hypodermic syringe, a pipette, and the like, for administering the compounds and compositions used in the practice of the invention.

### Compounds of the Invention

### Formula I

In one embodiment, the compounds of the present invention are represented by Formula I:

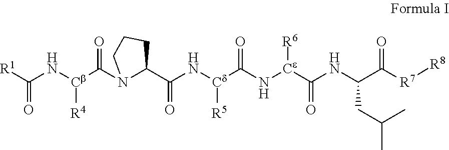

wherein:

R¹ is H, alkyl, cycloalkyl, aryl, heteroaryl, heterocyclic or C^{α}HR²R³;

R² and R⁴ are independently —(CH₂)ₘNR⁸R⁹R¹⁰, —(CH₂)ₘNR⁹C(═NR⁹)NR⁹R¹⁰, or —(CH₂)ₘ-imidazolidin-2-imin-1-yl;

R³ is —NR⁸R⁹R¹⁰, —N(R⁹)—C(═O)R⁹, —C^{φ}HR⁹R¹⁰, —C^{φ}H(R⁹)—C(═O)R¹⁰, or —C^{φ}H(C(═O)R⁹)(C(═O)R¹⁰);

R⁵ is phenyl, benzyl, —CH₂-(4-hydroxy-phenyl), —CH₂— (indol-3-yl), —C₁₋₁₂-(indol-4-yl), —CH₂-(napht-1-yl), —CH₂-(napht-2-yl), —CH₂— (aryl), —CH₂-(heteroaryl), napht-1-yl, or napht-2-yl;

R⁶ is methyl, ethyl, propyl, isopropyl, n-butyl, i-butyl, t-butyl, (2S)-butyl, (2R)-butyl, C₅₋₆ alkyl, cyclopropyl, cyclopropylmethyl, cyclopentyl, cyclohexyl, cyclopentylmethyl, or cyclohexylmethyl;
R⁷ is —O— or —N(R⁹)—;

R⁸, R⁹ and R¹⁰ are, independently in each instance, H, alkyl, cycloalkyl, aryl, heteroaryl, heterocyclic, or (CH₂CH₂O)ₙCH₃;
m is 1, 2, 3, 4 or 5;
n is an integer of from 1 to 20; and

C^{β}, C^{γ}, C^{ε} and C^{φ} are carbon atoms, and the stereochemistries at C^{α}, C^{β}, C^{γ}, C^{ε} and C^{φ} are independently either R or S; or any acceptable salt thereof.

In a preferred aspect, the compound of the invention is selected from the group consisting of ABS-295, ABS-296, ABS-298, ABS-334, ABS-357, ABS-358, ABS-359, ABS-363, ABS-368, ABS-398 and ABS-399. In another preferred aspect, the compound of the invention is ABS-363.

### Compounds of the Invention

### Formula II

In one embodiment, the compounds of the present invention are represented by Formula II:

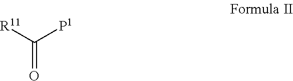

wherein:

P¹ is a known or novel peptide;

R¹¹ consists of one of Formulas IIa, IIb, IIc and IId, wherein the N-terminus amine group of P¹ is covalently coupled to the R¹¹C(O)— group through a peptide bond, wherein:

(a) Formula IIa is:

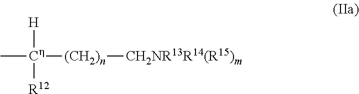

wherein
   n is 0, 1, 3, 4, or 5;
   m is zero or an integer of 1;
   R¹² is hydrogen, a straight or branched chain alkyl group of C₁-C₁₀, a cycloalkyl group of C₃-C₆, an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination;
   R¹³, R¹⁴, and R¹⁵ are, independently, hydrogen or branched or straight chain alkyl, alkenyl or alkynyl of C₁-C₁₀, an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination and with the proviso that a maximum of two of R¹³, R¹⁴ and R¹⁵ may be selected to be the aromatic, substituted aromatic, heteroaromatic or substituted heteroaromatic group; and
   C^{η} is a carbon atom and the stereochemistry at C is either R or S;

(b) Formula IIb is:

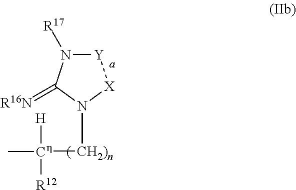

wherein
   n is an integer of from 0 to 6;
   when dashed line a is not present, X and Y are, independently, hydrogen or lower branched or straight chain alkyl, alkenyl or alkynyl of C₁-C₆;
   when dashed line a is present, X—Y is (CH₂)_{z}, wherein z is an integer of from 1 to 8;
   R¹² is hydrogen, a straight or branched chain alkyl group of C₁-C₁₀, a cycloalkyl group of C₃-C₆, an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination;
   R¹⁶ and R¹⁷ are independently hydrogen, lower branched or straight chain alkyl of C₁-C₁₀, lower branched or straight chain alkenyl of C₁-C₁₀, lower branched or straight chain alkynyl of C₁-C₁₀, an aromatic group of C₆-C₁₈ or a corresponding substituted aromatic group with one or two substituents selected from halogen, alkyloxy, carboxy, amide or alkyl in any combination, a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination;
   C^{η} is a carbon atom and the stereochemistry at C is either R or S;

(c) Formula IIc is:

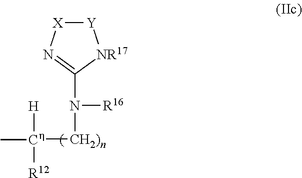

wherein
   n is an integer of from 0 to 5;
   X—Y is (CH₂)_{z}, wherein z is an integer of from 0 to 6;
   R¹² is hydrogen, a straight or branched chain alkyl group of C₁-C₁₀, a cycloalkyl group of C₃-C₆, an aromatic group of C₆-C₁₈ or a corresponding substituted aromatic group with one or two substituents selected from halogen, alkyloxy, carboxy, amide or alkyl in any combination, or a heteroaromatic group of C₄-C₈ and one or two heteroatoms selected from oxygen, sulfur and nitrogen in any combination or a corresponding substituted heteroaromatic group with one or two substituents selected from halogen, alkyloxy, carboxy, amide or alkyl in any combination;
   R¹⁶ and R¹⁷ are independently hydrogen, lower branched or straight chain alkyl of C₁-C₁₀, lower branched or straight chain alkenyl of C₁-C₁₀, lower branched or straight chain alkynyl of C₁-C₁₀, an aromatic group of C₆-C₁₈ or a corresponding substituted aromatic group with one or two substituents selected from halogen, alkyloxy, carboxy, amide or alkyl in any combination, a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination;
   C_{η} is a carbon atom and the stereochemistry at C is either R or S; and,

(d) Formula IId is:

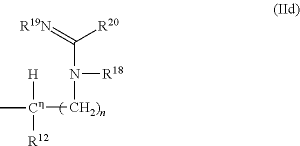

wherein
   n is an integer of from 0 to 5;
   R¹² is hydrogen, a straight or branched chain alkyl group of C₁-C₁₀, a cycloalkyl group of C₃-C₆, an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination;
   R¹⁸, R¹⁹, and R²⁹ are, independently, hydrogen or lower branched or straight chain alkyl, a cycloalkyl group of C₃-C₆, alkenyl or alkynyl of C₁-C₁₀, an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination, with the proviso that a maximum of two of and R¹⁸, R¹⁹, R²⁰ may be selected to be the aromatic, substituted aromatic, heteroaromatic or substituted heteroaromatic group; and
   C^{α} is a carbon atom and the stereochemistry at C is either R or S; or any acceptable salt thereof.

### Compounds of the Invention

### Neo-Trp-Containing Peptides

In one embodiment, the compounds of the present invention include the neo-Trp-containing peptides represented by compounds PP-1, PP-2, PP-3, PP-4, PP-5, PP-6, PP-7, PP-8, PP-9, PP-10, PP-11, PP-12, PP-13, PP-14, PP-15, PP-16, PP-17 and PP-18. Herein, the term “neo-Trp-containing peptide” refers to one or more of the compounds above (PP-1 through PP-18). The sequences for compounds PP-1 through PP-18 are shown in FIG. 7.

### Synthesis of Compounds of the Invention Encompassing Formula I, Formula II and Neo-Trp-Containing Peptides

### Subunits.

The compounds of the invention may be prepared by coupling of individual subunits. For example, in a compound of Formula I where R¹ is methyl, R⁴ is —(CH₂)₄NH₂, R⁵ is benzyl, R⁶ is sec-butyl, R⁷ is —O— and R⁸ is H, the subunits could be construed as being acetic acid, lysine, proline, phenylalanine, isoleucine and leucine, as shown below. This example only illustrates the possibility of envisioning the compound of Formula I as being composed of different subunits and should be not considered to introduce any limitation or preference in this disclosure.

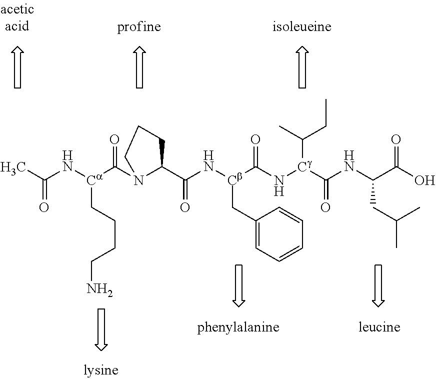

The compounds of Formula II and the neo-Trp-containing peptides may also be envisioned as being composed of different subunits. Therefore, the following discussion applies to compounds of Formula I, compounds of Formula II and neo-Trp-containing peptides.

One skilled in the art would recognize that the compounds of Formula I, compounds of Formula II or neo-Trp-containing peptides are composed of subunits connected by amide bonds. Therefore, such compounds could be prepared by formation of amide bonds between the various subunits. Each subunit may be an amino acid, an acid or an amine.

The subunits may contain substituents on their side chains, and such substituents may include amino groups, carboxylate groups or arginino groups, for example. One skilled in the art would appreciate that such side chain groups may be protected with a protective group before creation of the amide group between the subunits. After formation of the amide group or at any later stage in the synthesis, the protective group of the side chain group may then be removed. See, for example, the review of amine protecting groups provided in “Compendium of Organic Synthetic Methods,” I&S Harrison, Wiley Interscience, New York, N.Y. (1971), the disclosure of which is incorporated herein by reference. For side chain amino groups, some of the preferred protective groups are Boc (tert-butoxy carbonyl) and Fmoc (fluorenylmethoxy carbonyl). These groups may be removed by treatment with acid, such as trifluoroacetic acid, or base, such as piperidine, respectively. The side chains of the subunits may also incorporate substituents that may be modified into other substituents at a later stage in the synthesis. For example, a nitro group in the side chain may be reduced to an amino group if so desired.

### Synthesis.

The subunits required for the synthesis of the compounds of the invention may be purchased from commercial sources or prepared by standard synthetic methods known to those skilled in the art. Designing appropriate synthetic routes for each subunit should not represent undue experimentation for those skilled in the art. Since the subunits will be covalently coupled to other subunits through amide bonds, it may be convenient to employ as starting materials conveniently protected subunits, which may be deprotected at a later stage of the synthesis. The protective group to be used should be selected so that it does not get cleaved under the coupling conditions but is cleaved under conditions mild enough to avoid decomposition of the compound of the invention or any intermediate in its synthesis. Synthetic routes to some of the subunits of interest are described in PCT Application No. WO 2006/009902, the disclosure of which is incorporated herein by reference in its entirety.

The subunits of the compounds of the invention may be, partially or in totality, assembled by the Merrifield solid phase method, which is an established method for preparing peptides to those skilled in the art (Merrifield, 1986, Science 232:341, the disclosure of which is incorporated herein by reference). Alternatively, the peptide minus one or more of the N-terminal units may be expressed recombinantly by known biological methods, and the final N-terminal residue or residues may be added by chemical methods or by enzymatic condensation with an aminopeptidase (“Enzyme Structure and Mechanism,” Alan Fersht, W.H. Freeman, New York, N.Y. (1985), the disclosure of which is incorporate herein by reference).

The Merrifield solid phase synthesis may be generically outlined as shown below. Starting with an appropriate anchor resin designed for amino group exposure, the carboxy terminus amino acid unit of the peptide having an amino protecting group such as an Fmoc group is anchored to the resin through a selectively cleavable carboxyl coupling link. The amino group of the anchored carboxy terminus unit is then deprotected, and the additional amino protected amino acid units are then sequentially coupled in proper sequence. Each coupling step involves deprotection of the protected amino group of the anchored peptide chain, followed by peptide condensation between that unprotected amino group and the carboxyl group of the next amino acid unit. The condensation may be obtained by carbodiimide coupling, by Schotten Bauman reaction or by activated acyl group condensation (“Advanced Organic Chemistry,” 4^{th} edition, J. March, Wiley InterScience, New York, N.Y. (1992)). Protection of amine, carboxyl or any other side chains using appropriate protecting groups that differ from the protecting groups of the α-amino group entering into the peptide condensation enables selective peptide condensation of the sequential amino acid units, Appropriate protection groups and conditions for solid phase peptide synthesis are known in the literature (Merrifield, 1986, Science 232:341).

As a non-limiting example of a solid-phase synthesis sequence that may be used to prepare the compounds of the invention, resin-bound N-α-Fmoc-leucine may be swelled in DMF (dimethylformamide) and then the Fmoc group may be cleaved with piperidine (20% in DMF), The piperidine solution may be removed with vacuum filtration and the resin-bound amino acid may be washed with DMF and methylene chloride. Amino acids (4 equivalents) may be activated in DMF using HOBt (N-hydroxy-benzotriazole, 4 equivalents), PyBOP (benzotriazol-1-yl-oxy-tripyrrolidinophosphonium hexafluorophosphate, 4 equivalents) and DIPEA (diisopropylethylamine, 10 equivalents) and added directly to the peptide reaction vessel. The amino acid couplings may be conducted for approximately 6 hours, the resin washed with DMF and methylene chloride and monitored for the presence of free amines using the Kaiser test (Kaiser et al., 1970, Anal. Biochem. 34:595-8). Residues may be recoupled if necessary. The procedure is repeated with subsequent amino acids to form the desired peptide. Acid-catalyzed deprotection may be performed with a trifluoroacetic acid (TFA) solution containing appropriate scavengers, and crude peptides may be precipitated in ice-cold ether.

Parts or the entirety of the peptide may also be produced by recombinant expression. This biological method involves reengineering a microbe to express parts or the entirety of the peptide. A DNA sequence encoding the sequence of parts or the entirety of the peptide may be inserted in proper reading form into a plasmid or other vector capable of causing microbial expression of the DNA. The vector may also contain appropriate control, promoter and selection DNA segments. Upon insertion into a microbe such as E. coli or B. subtilus, the microbe mixture may be selected for appropriate transfection by treatment with the corresponding selection agent. Typically the agent is an antibiotic, and the vector contains a sequence encoding the corresponding detoxifying enzyme for the antibiotic (such as chloramphenacol or penicillin). Culturing the transfected microbe and harvesting the expressed peptide, as either secreted material of the culture medium or by lysing the microbe cells, provides the crude peptide. The peptide may be purified by known techniques such as lyophilization, chromatography (such as reverse phase high pressure liquid chromatography) and the like. These recombinant techniques for peptide expression are fully set forth in “Cold Spring Harbor—Current Protocols in molecular Biology”, Wiley InterScience, Cold Spring Harbor (2003), the disclosure of which is incorporated herein by reference.

### Biological Activity:

### Hypothermia as a Preliminary Screen of CNS Activity

Neurotensin (NT) induces hypothermia when administered directly into the CNS, possibly due to its actions at NTR₁. Likewise, NT(8-13) analog peptides that cross the blood-brain barrier after peripheral administration and have activity in the NT receptors also cause hypothermia. Therefore, one possible manner to determine whether the compounds of the invention are capable of interacting with the CNS is to monitor the body temperature of the subject after administration of the compounds. A significant hypothermic effect would demonstrate that the peptide has sufficiently good blood stability and membrane crossing ability to elicit a biological response. A compound that does not cross the blood-brain barrier (BBB) and/or does not penetrate the CNS would not be expected to influence body temperature.

Intraperitoneal injection is the standard route of administration to determine the extent of BBB crossing of neurotensin analogues. The methods and protocols are provided in PCT Application No WO 2006/009902, incorporated herein in its entirety. Intravenous administration results in a dose that is completely available to the systemic circulation. By contrast, an intraperitoneal injection is a more rigorous test of stability because the peptide is exposed to first pass metabolism in the liver.

### Schizophrenia Investigation

The blockade of locomotion caused by d-amphetamine (DA), a “DA agonist,” is the standard measure of therapeutic efficacy of current or potential drug candidates for treatment of schizophrenia. This model operates on the assumption that the direct stimulation of DA receptors within the mesolimbic DA system is responsible for the locomotor response. NT(8-13) analogues currently under investigation have demonstrated the ability to decrease DA induced hyperactivity in a dose-dependent fashion. Sound- and light-attenuated locomotor cages are used to measure the ability of potential candidates to decrease d-amphetamine-induced hyperactivity.

Catalepsy, commonly defined as a state of tonic immobility in rodents, is regarded as analogous to EPSEs (extrapyramidal side effects) in humans. Consequently, catalepsy is a side effect to be avoided in a successful drug candidate. Concurrently, the degree to which a drug candidate causes catalepsy in rats may also be used as a predictor for the probable occurrence of EPSEs associated with that particular candidate.

In laboratory tests, catalepsy is characterized by the inability of an animal to correct its position after placement in an unnatural posture. Catalepsy tests may be greatly influenced by a number of variables. These include stress-induced inhibition of catalepsy caused by a new environment and the contribution of learned “pseudo-catalepsy” that may result upon repeated measures with the same animal. To circumvent these potential confounding factors, tests are performed on an animal only once in a quiet, controlled environment,

### Pharmaceutical Compositions

When used in vivo, the compounds of the invention are preferably administered as a pharmaceutical composition, comprising a compound of the invention and a pharmaceutically acceptable carrier. The compounds of the invention may be present in a pharmaceutical composition in an amount from 0.001 to 99.9 wt %, more preferably from about 0.01 to 99 wt %, and even more preferably from 0.1 to 95 wt %.

Single or multiple administrations of the compositions may be administered depending on the dosage and frequency as required and tolerated by the subject. In any event, the administration regime should provide a sufficient quantity of the composition of this invention to treat the subject effectively.

All of the various compounds of the invention to be administered need not be administered together in a single composition. The different compounds may be administered in separate compositions. For example, if three different compounds of the invention are to be administered, the three different compounds may be delivered in three separate compositions. In addition, each compound may be delivered at the same time, or the compounds may be delivered consecutively with respect to one another. Thus, the mixture of the compounds of the invention may be administered in a single composition, or in multiple compositions comprising one or more compounds of the invention.

A mixture of two or more compounds of the invention may be administered in equimolar concentrations to a subject in need of such treatment. In another instance, two or more of the compounds of the invention are administered in concentrations that are not equimolar. In other instances, two or more of the compounds of the invention are administered as equal amounts, by weight, per kilogram of body weight. For example, the compounds of the invention may be administered in equal amounts, based on the weight of the subject. In another instance, the compounds of the invention are administered in unequal amounts. In yet other instances, the amount of each compound of the invention to be administered is based on its biological activity. In general, the schedule or timing of administration of a pharmaceutical composition of the invention is according to the accepted practice for the procedure being performed.

The regimen of administration may affect what constitutes an effective amount. For example, the therapeutic formulations may be administered to the subject either prior to or after the onset of a shivering, temperature spiking, pain or psychosis event. Further, several divided dosages, as well as staggered dosages, may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

In view of the disclosure contained herein, those skilled in the art will appreciate that the present compositions are capable of having a beneficial effect in a variety of surgical interventions, such as, but not limited to, general surgery, cosmetic surgery, neurosurgical procedures, cardiovascular procedures, organ amputations, and oncology-related interventions. It is therefore contemplated that the compositions of this invention may take numerous and varied forms, depending upon the particular circumstance of each application. For example, the compounds of the invention may be incorporated into a solid pill or may in the form of a liquid dispersion or suspension. In general, therefore, the compositions of the present invention preferably comprise a compound of Formula I, compound of Formula II or neo-Trp-containing peptide and a suitable, non-toxic, physiologically acceptable carrier. The compounds of the invention may be administered by any method designed to allow compounds to have a physiological effect. Administration may occur enterally or parenterally; for example orally, rectally, intracisternally, intravaginally, intraperitoneally or locally. Parenteral and local administrations are preferred.

For some applications involving treatment of shivering or temperature spiking in the broadest sense, it is desirable to have available a physically applicable or implantable predetermined solid form of material containing the composition of the invention. In such embodiments, the compositions of the present invention are preferably combined with a solid carrier that itself is bio-acceptable and suitably shaped for its use.

Formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Pharmaceutical compositions that are useful in the methods used in the practice of the invention may be prepared, packaged, or sold in formulations suitable for oral, rectal, intracisternal, intravaginal, intraperitoneal or local, or another route of administration. Other contemplated formulations include projected nanoparticles, liposomal preparations, resealed erythrocytes containing the active ingredient, and immunologically-based formulations.

The pharmaceutical compositions of the invention may be dispensed to the subject under treatment with the help of an applicator. The applicator to be used may depend on the specific medical condition being treated, amount and physical status of the pharmaceutical composition, and choice of those skilled in the art.

The pharmaceutical compositions of the invention may be provided to the subject or the medical professional in charge of dispensing the composition to the subject, along with instructional material. The instructional material includes a publication, a recording, a diagram, or any other medium of expression, which may be used to communicate the usefulness of the composition and/or compound used in the practice of the invention in a kit. The instructional material of the kit may, for example, be affixed to a container that contains the compound and/or composition used in the practice of the invention or shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

### Oral Administration.

A formulation of a pharmaceutical composition used in the practice of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion. As used herein, an “oily” liquid comprises a carbon-containing liquid molecule that exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture.

Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets, Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition used in the practice of the invention that are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropyl methylcellulose. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Powdered and granular formulations of a pharmaceutical preparation used in the practice of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition used in the practice of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination thereof. The compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (e.g. such as with a physiologically degradable material), and methods of absorbing an aqueous or oily solution or suspension into an absorbent material, with or without subsequent drying.

### Parenteral Administration.

As used herein, “parenteral administration” of a pharmaceutical composition includes any route of administration characterized by physical breaching of a tissue of a subject and administration of the pharmaceutical composition through the breach in the tissue. For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulator), agents such as suspending, stabilizing and/or dispersing agents, may be used. Such formulations may further comprise one or more additional ingredients including, but not limited to, suspending, stabilizing, or dispersing agents. In one embodiment of a formulation for parenteral administration, the active ingredient is provided in dry (e.g. powder or granular) form for reconstitution with a suitable vehicle (e.g. sterile pyrogen free water) prior to parenteral administration of the reconstituted composition.

Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques. Particularly preferred parenteral administration methods include intravascular administration (e.g., intravenous bolus injection, intravenous infusion, intra-arterial bolus injection, intra-arterial infusion and catheter instillation into the vasculature), peri- and intratarget tissue injection, subcutaneous injection or deposition including subcutaneous infusion (such as by osmotic pumps), intramuscular injection, intraperitoneal injection, and direct application to the target area, for example by a catheter or other placement device.

For parenteral administration, the compositions for administration may commonly comprise a solution or suspension of the compound in a pharmaceutically acceptable carrier, preferably an aqueous carrier. A variety of aqueous carriers may be used, e.g., buffered saline and the like. These suspensions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The amount of the compound may vary widely, and will be selected primarily based on fluid volumes, viscosities, body weight and the like in accordance with the particular mode of administration selected and the subject's needs. A typical pharmaceutical composition for intravenous administration would be about 1 to 3,000 mg per subject per day. Dosages from 1 up to about 1,000 mg per subject per day may be used, particularly when the drug is administered to a secluded site and not into the blood stream, such as into a body cavity or into a lumen of an organ. Methods for preparing parenterally administrable compositions will be known or apparent to those skilled in the art and are described in more detail in such publications as Remington's Pharmaceutical Science, 15th ed., 1980, Mack Publishing Company, Easton (PA).

### Transmucosal Administration.

Transmucosal administration is carried out using any type of formulation or dosage unit suitable for application to mucosal tissue. For example, the selected active agent may be administered to the buccal mucosa in an adhesive tablet or patch, sublingually administered by placing a solid dosage form under the tongue, lingually administered by placing a solid dosage form on the tongue, administered nasally as droplets or a nasal spray, administered by inhalation of an aerosol formulation, a non-aerosol liquid formulation, or a dry powder, placed within or near the rectum (“transrectal” formulations), or administered to the urethra as a suppository, ointment, or the like.

### Transurethal Administration.

With regard to transurethal administration, the formulation may comprise a urethral dosage form containing the active agent and one or more selected carriers or excipients, such as water, silicone, waxes, petroleum jelly, polyethylene glycol (“PEG”), propylene glycol (“PG”), liposomes, sugars such as mannitol and lactose, and/or a variety of other materials. A transurethral permeation enhancer may be included in the dosage from. Examples of suitable permeation enhancers include dimethylsulfoxide (“DMSO”), dimethyl formamide (“DMF”), N,N-dimethylacetamide (“DMA”), decylmethylsulfoxide (“C10 MSO”), polyethylene glycol monolaurate (“PEGML”), glycerol monolaurate, lecithin, the 1-substituted azacycloheptan-2-ones, particularly 1-n-dodecyl-cyclazacycloheptan-2-one (available under the trademark Azone™ from Nelson Research & Development Co., Irvine, Calif.), SEPA™ (available from Macrochem Co., Lexington, Mass.), surfactants as discussed above, including, for example, Tergitol™, Nonoxyno 1-9™ and TWEEN-80™, and lower alkanols such as ethanol.

### Transrectal Administration.

Transrectal dosage forms may include rectal suppositories, creams, ointments, and liquid formulations (enemas). The suppository, cream, ointment or liquid formulation for transrectal delivery comprises a therapeutically effective amount of the selected active agent and one or more conventional nontoxic carriers suitable for transrectal drug administration. The transrectal dosage forms of the present invention may be manufactured using conventional processes. The transrectal dosage unit may be fabricated to disintegrate rapidly or over a period of several hours. The time period for complete disintegration may be in the range of from about 10 minutes to about 6 hours, e.g., less than about 3 hours,

### Vaginal or Perivaginal Administration.

Vaginal or perivaginal dosage forms may include vaginal suppositories, creams, ointments, liquid formulations, pessaries, tampons, gels, pastes, foams or sprays. The suppository, cream, ointment, liquid formulation, pessary, tampon, gel, paste, foam or spray for vaginal or perivaginal delivery comprises a therapeutically effective amount of the selected active agent and one or more conventional nontoxic carriers suitable for vaginal or perivaginal drug administration. The vaginal or perivaginal forms of the present invention may be manufactured using conventional processes as disclosed in Remington: The Science and Practice of Pharmacy, supra (see also drug formulations as adapted in U.S. Pat. Nos. 6,515,198; 6,500,822; 6,417,186; 6,416,779; 6,376,500; 6,355,641; 6,258,819; 6,172,062; and 6,086,909). The vaginal or perivaginal dosage unit may be fabricated to disintegrate rapidly or over a period of several hours. The time period for complete disintegration may be in the range of from about 10 minutes to about 6 hours, e.g., less than about 3 hours.

### Intranasal or Inhalation Administration.

The active agents may also be administered intranasally or by inhalation. Compositions for intranasal administration are generally liquid formulations for administration as a spray or in the form of drops, although powder formulations for intranasal administration, e.g., insufflations, nasal gels, creams, pastes or ointments or other suitable formulators may be used. For liquid formulations, the active agent may be formulated into a solution, e.g., water or isotonic saline, buffered or unbuffered, or as a suspension. In certain embodiments, such solutions or suspensions are isotonic relative to nasal secretions and of about the same pH, ranging e.g., from about pH 4.0 to about pH 7.4 or, from about pH 6.0 to about pH 7.0. Buffers should be physiologically compatible and include, for example, phosphate buffers. Furthermore, various devices are available in the art for the generation of drops, droplets and sprays, including droppers, squeeze bottles, and manually and electrically powered intranasal pump dispensers. Active agent containing intranasal carriers may also include nasal gels, creams, pastes or ointments with a viscosity of, e.g., from about 10 to about 6,500 cps, or greater, depending on the desired sustained contact with the nasal mucosal surfaces. Such carrier viscous formulations may be based upon, for example, alkylcelluloses and/or other biocompatible carriers of high viscosity well known to the art (see e.g., Remington: The Science and Practice of Pharmacy, supra). Other ingredients, such as preservatives, colorants, lubricating or viscous mineral or vegetable oils, perfumes, natural or synthetic plant extracts such as aromatic oils, and humectants and viscosity enhancers such as, e.g., glycerol, may also be included to provide additional viscosity, moisture retention and a pleasant texture and odor for the formulation. Formulations for inhalation may be prepared as an aerosol, either a solution aerosol in which the active agent is solubilized in a carrier (e.g., propellant) or a dispersion aerosol in which the active agent is suspended or dispersed throughout a carrier and an optional solvent. Non-aerosol formulations for inhalation may take the form of a liquid, typically an aqueous suspension, although aqueous solutions may be used as well. In such a case, the carrier is typically a sodium chloride solution having a concentration such that the formulation is isotonic relative to normal body fluid. In addition to the carrier, the liquid formulations may contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Non-aerosol formulations for inhalation may also comprise dry powder formulations, particularly insufflations in which the powder has an average particle size of from about 0.1 μm to about 50 gm, e.g., from about 1 μm to about 25 μm.

Topical Formulations.

Topical formulations may be in any form suitable for application to the body surface, and may comprise, for example, an ointment, cream, gel, lotion, solution, paste or the like, and/or may be prepared so as to contain liposomes, micelles, and/or microspheres. In certain embodiments, topical formulations herein are ointments, creams and gels.

### Transdermal Administration.

Transdermal compound administration, which is known to one skilled in the art, involves the delivery of pharmaceutical compounds via percutaneous passage of the compound into the systemic circulation of the subject. Topical administration may also involve the use of transdermal administration such as transdermal patches or iontophoresis devices. Other components may be incorporated into the transdermal patches as well. For example, compositions and/or transdermal patches may be formulated with one or more preservatives or bacteriostatic agents including, but not limited to, methyl hydroxybenzoate, propyl hydroxybenzoate, chlorocresol, benzalkonium chloride, and the like. Dosage forms for topical administration of the compounds and compositions may include creams, sprays, lotions, gels, ointments, eye drops, nose drops, ear drops, and the like. In such dosage forms, the compositions of the invention may be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the compositions may contain polyethylene glycol 400. They may be mixed to form ointments with, for example, benzyl alcohol 2% (wt/wt) as preservative, white petrolatum, emulsifying wax, and tenox II (butylated hydroxyanisole, propyl gallate, citric acid, propylene glycol). Woven pads or rolls of bandaging material, e.g., gauze, may be impregnated with the compositions in solution, lotion, cream, ointment or other such form may also be used for topical application. The compositions may also be applied topically using a transdermal system, such as one of an acrylic-based polymer adhesive with a resinous crosslinking agent impregnated with the composition and laminated to an impermeable backing.

Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir that, in this case, may be either a polymeric matrix as described above, or be a liquid or hydrogel reservoir, or take some other form.

### Intrathecal Administration.

One common system utilized for intrathecal administration is the APT Intrathecal treatment system available from Medtronic, Inc. APT Intrathecal uses a small pump that is surgically placed under the skin of the abdomen to deliver medication directly into the intrathecal space. The medication is delivered through a small tube called a catheter that is also surgically placed. The medication may then be administered directly to cells in the spinal cord involved in conveying sensory and motor signals associated with lower urinary tract disorders.

### Intravesical Administration.

The term intravesical administration is used herein in its conventional sense to mean delivery of a drug directly into the bladder. Suitable methods for intravesical administration may be found, for example, in U.S. Pat. Nos. 6,207,180 and 6,039,967.

### Additional Administration Forms.

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Application Nos. 2003/0147952, 2003/0104062, 2003/0104053, 2003/0044466, 2003/0039688, and 2002/0051820. Additional dosage forms of this invention also include dosage forms as described in PCT Application Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the formulations of the present invention may be, but are not limited to, short-term release or rapid-offset release, as well as controlled release, for example, sustained release, delayed release and pulsatile release formulations.

The term short-term or rapid-offset release is used in its conventional sense to refer to a drug formulation that provides for release of the drug immediately after drug administration.

As used herein, short-term or rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments there between after drug administration after drug administration.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be longer than the time required for the release of the same amount of agent administered in bolus form.

For sustained release, the compounds may be formulated with a suitable polymer or hydrophobic material that provides sustained release properties to the compounds. As such, the compounds of the invention may be administered in the form of microparticles for example, by injection or in the form of wafers or discs by implantation.

In a preferred embodiment of the invention, the compounds of Formula I or compounds of Formula II or neo-Trp-containing peptides are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

In a preferred embodiment of the invention the compounds of Formula I or compounds of Formula II or neo-Trp-containing peptides are administered to a subject, alone or in combination with another pharmaceutical agent, using a delayed release formulation.

The term pulsatile release is used herein in its conventional sense to refer to a drug formulation that provides release of the drug in such a way as to produce pulsed plasma profiles of the drug after drug administration.

In a preferred embodiment of the invention, the compounds of Formula or compounds of Formula II or neo-Trp-containing peptides are administered to a subject, alone or in combination with another pharmaceutical agent, using a pulsatile release formulation.

### Dosing

One skilled in the art may readily determine an effective amount of compounds of the invention to a given subject, by taking into account factors such as the size and weight of the subject; the extent of shivering or temperature spiking observed in the subject or the amount of pain endured by the subject or the degree of psychosis experienced by the subject or the lowering of body temperature desired for the subject; the age, health and sex of the subject; the route of administration; and whether the administration is local or systemic. Generally, the amount of compounds of the invention to be administered to a subject depends upon the degree of shivering or temperature spiking or pain or psychosis, and the biological activity exhibited by the compounds of the invention. Those skilled in the art may derive appropriate dosages and schedules of administration to suit the specific circumstances and needs of the subject. For example, suitable doses of compounds of the invention to be administered may be between about 0.015 mg/kg and about 50 mg/kg body weight. In some embodiments, dosages are between about 0.1 mg/kg and about 20 mg/kg body weight.

It is understood that the effective dosage will depend on the age, sex, health, and weight of the subject, kind of concurrent treatment, if any, frequency of treatment, and the nature of the effect desired. The most preferred dosage will be tailored to the individual subject, as is understood and determinable by one of skill in the art, without undue experimentation.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

A suitable dose of a compound of the present invention may be in the range of from about 1 mg to about 5,000 mg per day, such as from about 10 mg to about 2,000 mg, for example. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12 hour interval between doses.

In some embodiments, dose of a compound of the invention is between about 1 mg and about 2,000 mg. In some embodiments, a dose of a compound of the invention used in compositions described herein is between about 2 mg and about 1,000 mg. In some embodiments, a dose of a compound of the invention used in compositions described herein is between about 4 mg and about 500 mg. In some embodiments, a dose of a compound of the invention used in compositions described herein is between about 8 mg and about 250 mg. In some embodiments, a dose of a compound of the invention used in compositions described herein is between about 16 mg and about 125 mg. In some embodiments, a dose of a compound of the invention used in compositions described herein is between about 30 mg and about 60 mg, and any and all whole or partial increments there between.

The amount of compound dosed per day may be administered every day, every other day, every 2 days, every 3 days, every 4 days, every 5 days, etc.

The pharmaceutical compositions for use in the method of the invention may be prepared, packaged, formulated or sold in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose. The specifications for the dosage unit forms of the invention are dictated by and directly dependent on: (a) the unique characteristics of the therapeutic compound and the particular therapeutic effect to be achieved, and (b) the limitations inherent in the art of compounding/formulating such a therapeutic compound for the treatment of shivering or temperature spiking in a subject.

### Combination Therapies and Treatments

The compounds of the present invention may be useful for the methods of the present invention in combination with mechanical procedures commonly used to treat, prevent, or reduce the symptoms of perioperative shivering or temperature spiking. Mechanical devices used for ameliorating, treating or preventing thermoregulation dysfunction in the context of an operative intervention include surgical drapes used for blanketing the subject, forced air warming systems, circulating water mattresses, radiant warmers and resistive heating blankets.

In one embodiment, the present invention provides a method of controlling, ameliorating or preventing shivering or temperature spiking associated with surgical intervention in a subject in need thereof. The method comprises administering to the subject an effective amount of the composition of the invention comprising a compound of Formula I and providing a mechanical procedure associated with the control of temperature spiking and shivering or temperature spiking. In another embodiment of this aspect of the present invention, the therapeutic effect achieved is synergistic, the therapeutic effect being greater than the sum of the therapeutic effect achieved by the administration of the compound of Formula I and providing the mechanical procedure separately. In another embodiment, the mechanical device comprises a surgical drape used for blanketing the subject, a forced air warming system, a circulating water mattress, a radiant warmer or a resistive heating blanket.

In another embodiment, the present invention provides a method of controlling, ameliorating or preventing shivering associated with surgical intervention in a subject in need thereof. The method comprises administering to the subject an effective amount of the composition of the invention comprising a compound of Formula II and providing a mechanical procedure associated with the control of temperature spiking and shivering or temperature spiking. In another embodiment of this aspect of the present invention, the therapeutic effect achieved is synergistic, the therapeutic effect being greater than the sum of the therapeutic effect achieved by the administration of the compound of Formula II and providing the mechanical procedure separately. In another embodiment, the mechanical device comprises a surgical drape used for blanketing the subject, a forced air warming system, a circulating water mattress, a radiant warmer or a resistive heating blanket.

In one embodiment, the present invention provides a method of controlling, ameliorating or preventing shivering associated with surgical intervention in a subject in need thereof. The method comprises administering to the subject an effective amount of the composition of the invention comprising a neo-Trp-containing peptide and providing a mechanical procedure associated with the control of temperature spiking and shivering or temperature spiking. In another embodiment of this aspect of the present invention, the therapeutic effect achieved is synergistic, the therapeutic effect being greater than the sum of the therapeutic effect achieved by the administration of the neo-Trp-containing peptide and providing the mechanical procedure separately. In another embodiment, the mechanical device comprises a surgical drape used for blanketing the subject, a forced air warming system, a circulating water mattress, a radiant warmer or a resistive heating blanket.

The compounds of the present invention may also be useful in the methods of the present invention, in combination with one or more additional compounds useful for treating perioperative shivering. The additional compound or compounds may comprise compounds of the present invention or other compounds known to treat, prevent, or reduce the symptoms of shivering or temperature spiking.

Drugs known to treat, prevent, or reduce perioperative shivering include opioids, alpha-2 agonists, serotonin neuromediators, corticosteroids and magnesium, among others. Non-limiting examples of opioids include pethidine (Demerol™, isonipecaine, lidol, pethanol, piridosal, Algil™, Alodan™, Centralgin™, Dispadol™, Dolantin™, Dolestine™, Dolosal™, Dolsin™, MefedinaAlgil™, Alodan™, Centralgin™) and alfentanil (Alfenta™: Janssen). Non-limiting examples of alpha-2 adrenergic agonists include clonidine (Catapres™, Dixaritand™) and dexmedetomidine (Precedex™: Hospira, Inc.). Non-limiting examples of serotonin neuromediators include tramadol (Tramal™, Ultram™: Grünenthal GmbH), Ketanserin™ (Janssen), nefopam (Acupan™) and ondansetron (Zofran™: GlaxoSmithKline). Other examples of drugs used to prevent or treat such conditions are methylphenidate (Ritalin™, Concerta™, Metadate™, Methylin™: Novartis), physostigmine (Antilirium™, Eserine™) and doxapram (Dopram™).

In one embodiment, the present invention provides a method of controlling, ameliorating or preventing shivering associated with surgical intervention in a subject in need thereof. The method comprising administering to the subject effective amounts of a combination of (a) a compound of Formula I, and (b) one or more additional compounds known to control, ameliorate or prevent shivering. The one or more additional compounds combined with the compound of Formula I may be selected from the group consisting of opioids, alpha-2 agonists, serotonin neuromediators, methylphenidate, physostigmine and doxapram. In an embodiment, the opioid is pethidine or alfentanil. In another embodiment, the alpha-2 agonist is clonidine or dexmedetomidine. In yet another embodiment, the serotonin neuromediator is tramadol, ketanserin, nefopam or ondansetron.

In another embodiment, the present invention provides a method of controlling, ameliorating or preventing shivering associated with surgical intervention in a subject in need thereof. The method comprises administering to the subject effective amounts of a combination of (a) a compound of Formula II, and (b) one or more additional compounds known to control, ameliorate or prevent shivering. The one or more additional compounds combined with the compound of Formula II may be selected from the group consisting of opioids, alpha-2 agonists, serotonin neuromediators, methylphenidate, physostigmine and doxapram. In an embodiment, the opioid is pethidine or alfentanil. In another embodiment, the alpha-2 agonist is clonidine or dexmedetomidine. In yet another embodiment, the serotonin neuromediator is tramadol, ketanserin, nefopam or ondansetron.

In yet another embodiment, the present invention provides a method of controlling, ameliorating or preventing shivering associated with surgical intervention in a subject in need thereof. The method comprises administering to the subject effective amounts of a combination of (a) a neo-Trp-containing peptide, and (b) one or more additional compounds known to control, ameliorate or prevent shivering. The one or more additional compounds combined with the neo-Trp-containing peptide may be selected from the group consisting of opioids, alpha-2 agonists, serotonin neuromediators, methylphenidate, physostigmine and doxapram. In an embodiment, the opioid is pethidine or alfentanil. In another embodiment, the alpha-2 agonist is clonidine or dexmedetomidine. In yet another embodiment, the serotonin neuromediator is tramadol, ketanserin, nefopam or ondansetron.

In an embodiment of this aspect of the present invention, the therapeutic effect achieved by the combination above is synergistic, in that, the therapeutic effect of the combination is greater than the sum of the therapeutic effect achieved by the separate administration of the compound of Formula I and the one or more compounds that are known to control, ameliorate or prevent shivering or temperature spiking.

In yet another embodiment of this aspect of the present invention, the therapeutic effect achieved by the combination above is synergistic, in that, the therapeutic effect of the combination is greater than the sum of the therapeutic effect achieved by the separate administration of the compound of Formula II and the one or more compounds that are known to control, ameliorate or prevent shivering or temperature spiking.

In yet another embodiment of this aspect of the present invention, the therapeutic effect achieved by the combination above is synergistic, in that, the therapeutic effect of the combination is greater than the sum of the therapeutic effect achieved by the separate administration of the neo-Trp-containing peptide and the one or more compounds that are known to control, ameliorate or prevent shivering or temperature spiking.

A combination of compounds described herein may either result in synergistic increase in effectiveness against shivering or temperature spiking, relative to effectiveness following administration of each compound when used alone, or such an increase may be additive. Compositions described herein typically include lower dosages of each compound in a composition, thereby avoiding adverse interactions between compounds and/or harmful side effects, such as ones that have been reported for similar compounds. Furthermore, normal amounts of each compound when given in combination could provide for greater efficacy in subjects who are either unresponsive or minimally responsive to each compound when used alone.

A synergistic effect may be calculated, for example, using suitable methods such as, for example, the Sigmoid-Eurax equation (Holford and Scheiner, 1981, Clin. Pharmacokinet. 6: 429-453), the equation of Loewe additivity (Loewe and Muischnek, 1926, Arch. Exp. Pathol Pharmacal. 114: 313-326) and the median-effect equation (Chou and Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials

Unless otherwise noted, all starting materials and resins were obtained from commercial suppliers and used without purification.

### Example 1

### Hypothermia Studies in the Monkey and Rat with ABS-212

Hypothermia studies in the monkey and the rat were conducted with ABS-212 at doses of 5-100 mg/kg. The compound was administered as a slow i.v. push over 20 to 30 seconds. Sedation was evident and occurred in a dose-dependent manner with maximum effect observed at 100 mg/kg. The animals became recumbent for up to 30 minutes, with full recovery. The no-effect dose was determined to be <10 mg/kg on day 1, with very mild sedation, Diminishing side effects of sedation and hypothermia were recorded on day 2 and day 3 in the monkey.

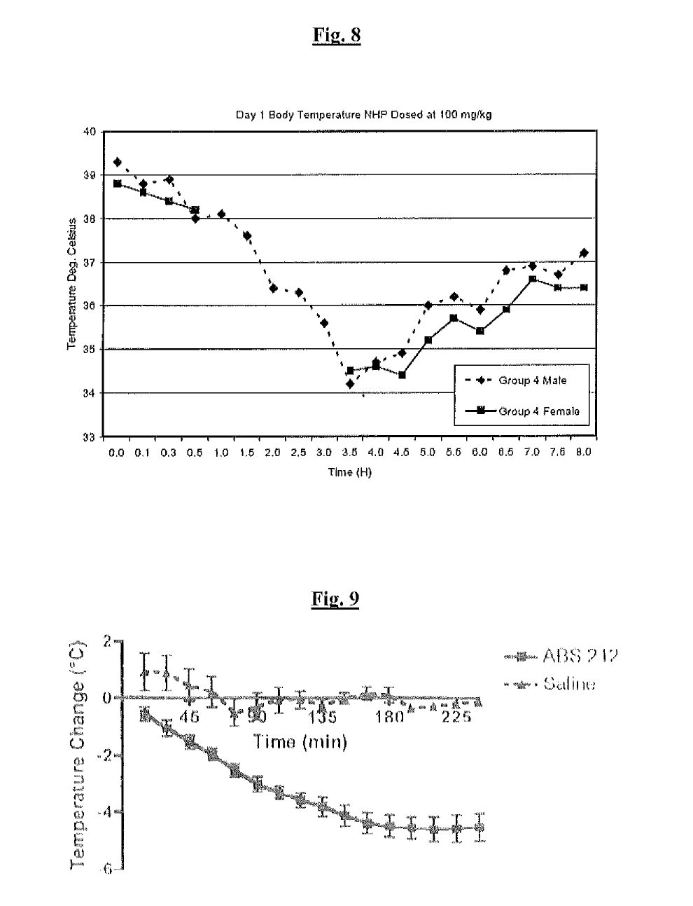

Hypothermia in the monkey was observed only at the high dose of 100 mg/kg, with decreases in temperature of up to 5° C. for 4 to 5 hours (FIG. 8). No signs of shivering or discomfort were noted, and hypothermia persisted in the presence of heat lamps and warm towels. The animals returned to normal temperature and activity within 20 hours post dose. Tolerance to induced hypothermia was noted after the second dose in monkeys.

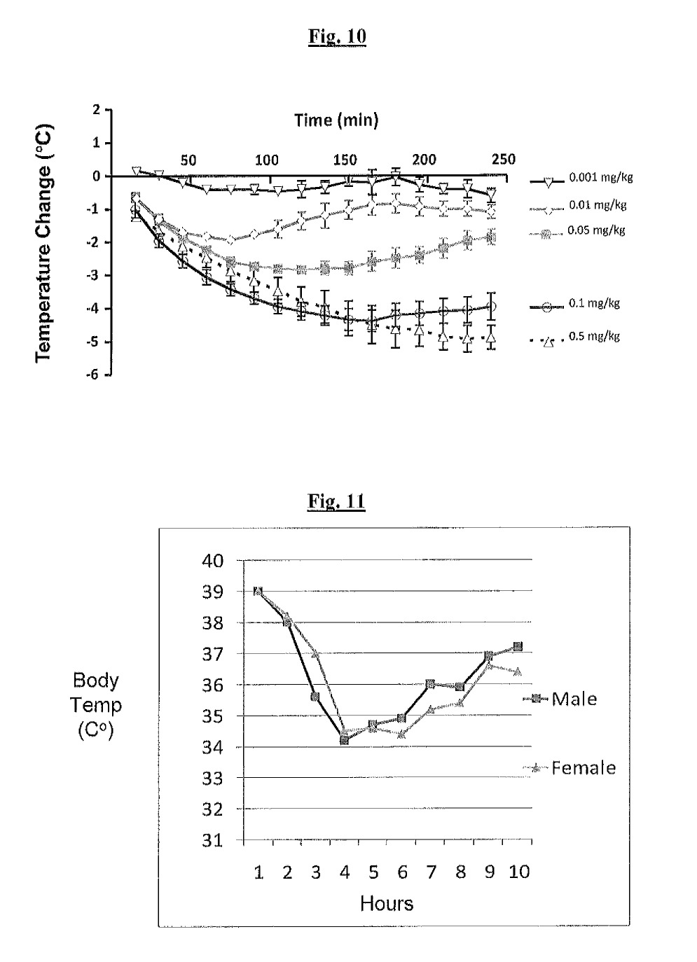

Rats reacted similarly to drug administration (FIG. 9), but no tolerance to hypothermia or analgesia was observed. All animals appeared normal within 20 hours of dosing.

### Example 2

### Hypothermia Studies in the Rat with Various Compounds

Hypothermia studies in the male Sprague-Dawley rat were conducted using different compounds (dosed in saline) and the results are summarized in Table 1. For each compound, the maximum decrease in temperature (Δtemp) and the area under the curve for hypothermia versus a saline control (AUC) were measured.

ABS-363 induced the greatest amount of hypothermia at a lower dose than the other compounds, and the hypothermia effect was dose-dependent (FIG. 10). ABS-363 was thus selected for further characterization studies.

TABLE 1

| | dose | | |
|---|---|---|---|
| Compound | (mg/kg) | Δtemp | AUC |
| ABS-363 | 0.5 | −4.9 | 817 |
| ABS-401 | 10 | −4.3 | 758 |
| ABS-357 | 10 | −4.2 | 724 |
| ABS-358 | 5 | −4.2 | 672 |
| SEQ ID NO:2 | 1 | −4.2 | 700 |
| ABS-377 | 5 | −4.2 | 691 |
| ABS-398 | 1 | −3.7 | 637 |
| ABS-359 | 10 | −3.4 | 665 |

### Example 3

### Hypothermia Studies in the Monkey with ABS-363

ABS-363 was intravenously injected into one male monkey and one female monkey at 0.1 mg/kg dose in saline, and the core body temperature for each animal was monitored using a rectal probe over time. As shown in FIG. 11, ABS-363 induced up to a 6° C. drop in monkeys, thus causing mild hypothermia without the use of forced cooling (ice, cooling jackets), nerve blocks to control shivering or intubation. The studies in rats described above suggest that this compound may be useful for controlling shivering.

### Example 4

### Hypothermic Analysis

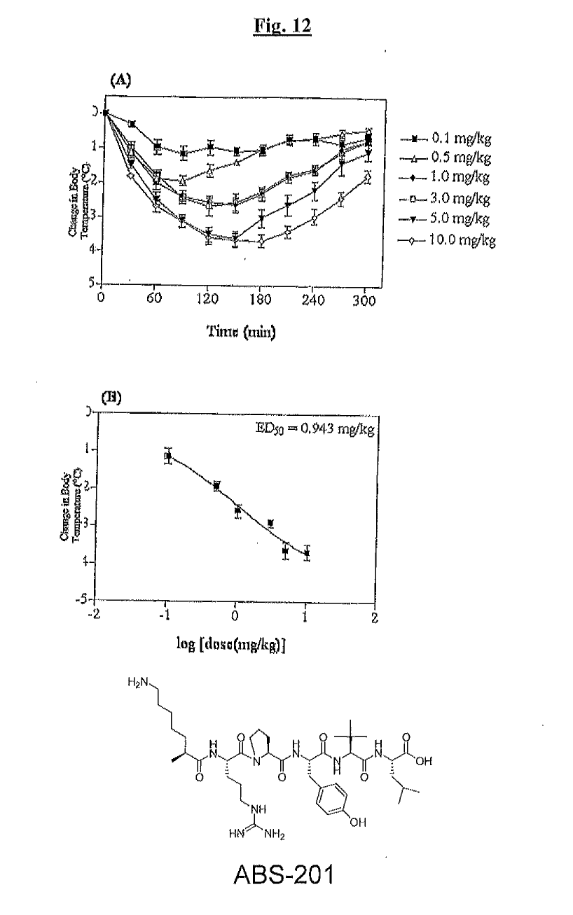

To examine the antipsychotic properties of ABS-201, a NT(8-13) analog which structure is shown in FIG. 12, a dose-response curve for hypothermic induction after i.p. administration was generated. The ability of ABS-201 to reduce d-amphetamine induced hyperlocomotion after i.p. administration also was measured. To assess the effects on CNS activity caused by prolonged treatment of ABS-201, hypothermia and attenuation of d-amphetamine induced hyperlocomotion were measured after repeated daily dosing. Finally, the bar test was utilized to measure catalepsy as a predictor of EPSEs in humans.

The dose-response curve for ABS-201 after i.p. injection over a concentration range of 0.1-10 mg/kg (FIG. 12b) showed that the maximal effect elicited at 5 mg/kg (−3.61±0.22° C. at 150 min PI, FIG. 12a) was a full degree greater than the maximal effect seen after the preliminary screen (−2.51±0.17° C. at 150 min PD. This discrepancy is most likely due to environmental factors (air temperature, rectal probes, rat size, etc.) that may affect the rats' response. Most importantly, ABS-201 continued to elicit a significant CNS effect irrespective of these differences. The ED₅₀ value for ABS-201, 0.943 mg/kg., compares favorably with other NT(8-13) analogues with CNS activity (Tyler-McMahon et al., 2000, Eur. J. Pharmacol. 390:107-111; Bottles et al., 2001, Brain Res. 919:1-11).

### Example 5

### Effects on d-Amphetamine-Induced Hyperactivity

The effects of ABS-201 on d-amphetamine induced hyperactivity at varying doses were also examined. ABS-201 significantly reduced hyperlocomotion for all doses tested (doses of 3 mg/kg and 10 mg/kg, data not shown). Another hallmark of current antipsychotic drugs is the ability to reduce spontaneous locomotor activity. All ABS-201 dose groups responded significantly lower than saline during the drug phase, indicating the ability of ABS-201 to reduce spontaneous activity.

Table 2 summarizes the hypothermic response to chronic i.p. administration of ABS-201 in rats. tₘₐₓ represented the time from administration to maximal temperature decrease, l.p. dose was 5 mg/kg for all days. ABS-201 maintained a significant CNS effect after repeated daily dosing in rats and over the 5-day period the absolute hypothermic response increased. A comparison of the induced hypothermia of ABS-201 on days 1 and 5 was made. On day 5, the maximal hypothermic response was achieved marginally faster (90 min) compared to day 1 (120 min). In contrast to day 1, on day 5 the maximal hypothermic effect was not maintained for an extended period, implying that while repeated dosing does not decrease the maximal effect, it may reduce the duration of the hypothermic effect, Repeated daily dosing had no effect on the ability of ABS-201 to attenuate d-amphetamine induced hyperlocomotion. Both the acute and chronic dosing groups produced a reduction in hyperactivity that was significant for almost two hours after amphetamine administration. Of note, chronic administration of ABS201 did abolish its inhibitory effect on spontaneous locomotor activity.

TABLE 2

| peptide | tₘₐₓ | decrease in body temperature |
|---|---|---|
| (N = 5) | (min) | measured at tₘₐₓ |
| saline | 180 | 0.70 ± 0.20 |
| Day 1 | 120 | 2.72 ± 0.24 |
| Day 2 | 90 | 2.85 ± 0.26 |
| Day 3 | 120 | 3.74 ± 0.13 |
| Day 4 | 120 | 3.71 ± 0.13 |
| Day 5 | 90 | 3.83 ± 0.24 |

### Example 6

### Cataleptic Analysis

Neither ABS-201 (5 mg/kg) nor saline caused catalepsy after peripheral administration. Haloperidol, a typical anti-schizophrenic drug known to produce a fully cataleptic response in rats, induced catalepsy that lasted for greater than 30 sec. These results demonstrate that ABS-201 does not induce catalepsy after peripheral administration, a hallmark of current clinically effective candidates.

### Example 7

### Anti-Shivering Studies

The anti-shivering properties of the compounds of the invention may be evaluated in rodent models, as described below. Two strategies may be used to induce shivering: (a) swimming in a cold water pool, and (b) recovery from isoflurane anesthesia.

### Example 7.1

### Swimming in a Cold Water Pool

In a preliminary experiment, rats (N=2) swam in a cold water pool (water temperature 20° C.) for 0, 15, 30, 60, 90 or 120 seconds and were then allowed to recover in a temperature controlled chamber (30° C.) for 20 minutes. A telemetric peritoneal temperature probe recorded the animal temperature before and after swimming, and for the twenty minutes during recovery after swimming. The exercising strategy was used because it consistently raises the shivering threshold (Kenny et al., 1999, Eur. J. Appl. Physiol. 79:495-499), and simulates the stress of surgery. Pilot experiments (N=2) revealed that shivering begins consistently at 35.5±0.2° C., which is approximately a 1° C. decrease in body temperature from baseline. In addition, the intensity of shivering and decrease in body temperature may be controlled by the duration of swimming. As the swimming duration increases, the intensity of shivering increases and the drop in body temperature also increases during the recovery period.

The decrease in body temperature during recovery from swimming ranged from −1±0.2° C. with 15 seconds of swimming to −3.4±0.4° C. with 120 seconds of swimming. Shivering was rated by visual inspection on a scale of 0-3, with 0=no shivering, 1=mild shivering, 2=moderate shivering and 3=severe shivering. The shivering scores (median) were performed by an investigator blinded to swimming duration and were: 0 at 0 seconds swim; 1 at 15 seconds; 2 at 30, 60 and 90 seconds; and 3 at 120 seconds.

### Example 7.2

### Recovery from Isoflurane Anesthesia

ABS-363 was evaluated in a postoperative surgery model using a single rat. In this protocol, the rat sustained 80 minutes of surgery with titrated isoflurane anesthesia. A core temperature probe was implanted in the rat abdomen, a femoral vein cannula was externalized, and an EMG line was inserted into the mid-back region. Then the rat is placed in a 30° C. high oxygen incubator for 210 minutes to regain temperature regulation while isoflurane dissipated. An ABS-363 injection (0.1 mg/kg, 1 ml/kg) was administered over a 10 minute period, 80 minutes prior to entering the shiver chamber. A saline injection (1 ml/kg) immediately followed the ABS-363 injection. After 210 minutes in the incubator, the rat was moved to the shiver chamber (walls temperature controlled). The rat remained in the shiver chamber for 60 minutes and then the wall temperature control was discontinued and the rat observed for another 60 minutes at ambient temperature (21° C.).

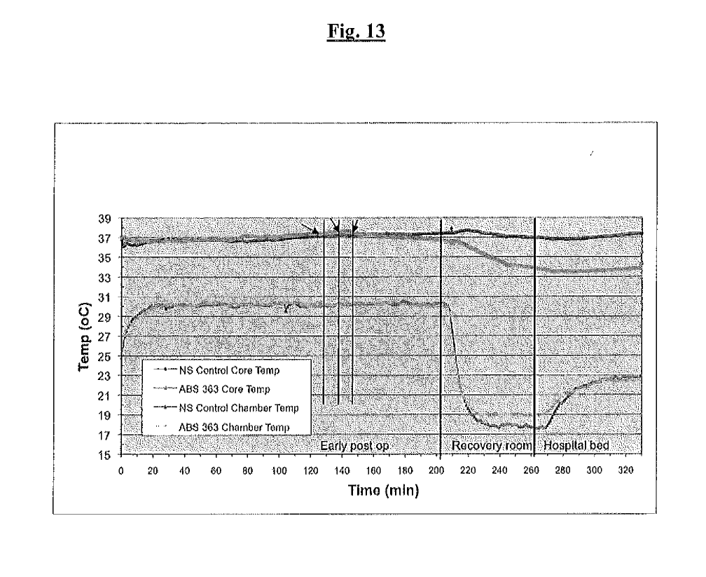

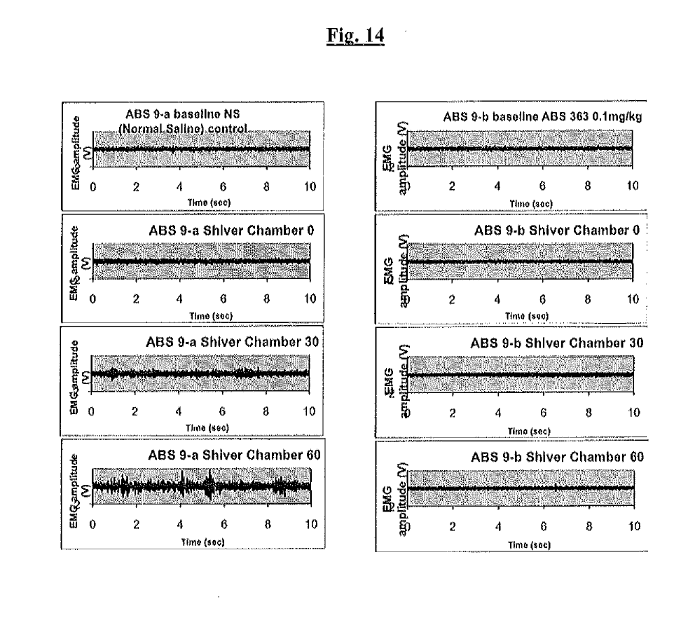

The time course of the experiment is shown in FIG. 13. Administration of ABS-363 induced a prolonged hypothermia effect versus saline control. The change in the shiver chamber temperature was the same for control and ABS-363 treated rats. As shown in FIG. 14, ABS-363 prevented shivering at all points evaluated after injection. Thus, ABS-363 transiently reset the temperature set point and blocked the shivering thermogenesis process at least to that new point. There was no rebound past the normothermic set point. Lower doses of ABS-363 should most likely block shivering while keeping the set point closer to normal.

### Example 7.3

### Research Design and Methods

The activity of the compounds of the invention may be determined in a randomized study.

### (a) Anesthesia Protocol:

Twenty Sprague-Dawley rats, weighing 275-300 grams, are equipped with tunneled venous cannulas and peritoneal telemetric probes with temperature and muscle activity monitoring capabilities (MiniMitter, Bend Oregon and Grass Instruments, Warwick R.I.), while under titrated isoflurane anesthesia. Rats are injected with 1 ml of study drug (saline or compound of the invention) at completion of isoflurane anesthesia and observed for one hour in a temperature controlled chamber. Body temperature and shivering (monitored by electromyography, also known as EMG) activity are recorded continuously throughout the one hour recovery period after surgery. An investigator blinded to therapeutic intervention records the onset and severity of shivering with the visual scale described above. Rats are then allowed to recover for a minimum of three days before the swimming experiments. The EMG tracings are also scored for shivering frequency and intensity by an investigator blinded to intervention.

### (b) Swim Protocol:

Rats are injected with 1 ml of study drug (normal saline or compound of interest) and observed at room temperature for one hour. Body temperature & EMG activity are recorded and downloaded to a computer for future analysis by an investigator blinded to study intervention. Rats then swim in a pool (water temperature 20° C.) for 30 seconds, and recover for 30 minutes in a temperature chamber at 30° C., where temperature & EMG activity are be recorded continuously and visually scored for shivering by an investigator blinded to study intervention. Rats undergo three swimming trials daily on two separate days with a minimum of one day for recovery between swimming trials. The temperature at onset of shivering, total decrease in temperature after study drug administration and the duration/intensity of shivering data downloaded to the computer are analyzed by an observer blinded to study intervention. Rats are then euthanized by an overdose of isoflurane, and organs are fixated with 2% paraformaldehyde for future histological analysis.

### (c) Statistical Analysis:

Shivering is defined as a 50% increase in EMG activity above baseline. A two tailed t-test and Chi-Square analysis are employed to evaluate temperature changes and presence of shivering. A logistic regression model is used to compare drug intervention, temperature change, onset and duration of shivering. Visual measures of shivering are compared between groups by Kruskal-Wallis analysis. Significance for all evaluations are set at p<0.05.

The disclosures of each and every patent, patent application, and publication cited herein are hereby incorporated herein by reference in their entirety. One skilled in the art will readily appreciate that the present invention is well adapted to carry out the objects and obtain the ends and advantages mentioned, as well as those inherent therein. While the invention has been disclosed with reference to specific embodiments, it is apparent that other embodiments and variations of this invention may be devised by others skilled in the art without departing from the true spirit and scope used in the practice of the invention. The appended claims are intended to be construed to include all such embodiments and equivalent variations.

1. A composition comprising a compound of Formula I: 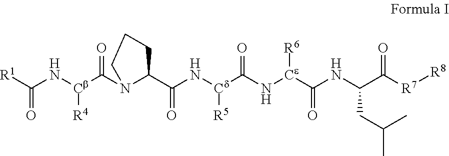
wherein:
R¹ is H, alkyl, cycloalkyl, aryl, heteroaryl, heterocyclic or C^{α}HR²R³;
R² and R⁴ are independently —(CH₂)ₘNR⁸R⁹R¹⁰, —(CH₂)ₘNR⁹C(═NR⁹)NR⁹R¹⁰, or —(CH₂)ₘ-imidazolidin-2-imin-1-yl;
R³ is —NR⁸R⁹R¹⁰, —N(R⁹)—C(═O)R⁹, —C^{φ}HR⁹R¹⁰, —OH(R⁹)—C(═O)R¹⁰, or —C^{φ}H(C(═O)R⁹)(C(═O)R¹⁰);
R⁵ is phenyl, benzyl, —CH₂-(4-hydroxy-phenyl), —CH₂— (indol-3-yl), —CH₂-(indol-4-yl), —CH₂-(napht-1-yl), —CH₂-(napht-2-yl), —CH₂-aryl, —CH₂-(heteroaryl), napht-1-yl, or napht-2-yl;
R⁶ is methyl, ethyl, propyl, isopropyl, n-butyl, i-butyl, t-butyl, (2S)-butyl, (2R)-butyl, C₅₋₆ alkyl, cyclopropyl, cyclopropylmethyl, cyclopentyl, cyclohexyl, cyclopentylmethyl, or cyclohexylmethyl;
R⁷ is —O— or —N(R⁹)—;
R⁸, R⁹ and R¹⁰ are, independently in each instance, H, alkyl, cycloalkyl, aryl, heteroaryl, heterocyclic, or (CH₂CH₂O)ₙCH₃;
m is 1, 2, 3, 4 or 5;
n is an integer of from 1 to 20; and
C^{α}, C^{β}, C^{γ}, C^{ε} and C^{φ} are carbon atoms, and the stereochemistries at C^{α}, C^{β}, C^{γ}, C^{ε} and C^{φ} are independently either R or S;
or any acceptable salt thereof.

2. The composition of claim 1, wherein said compound is selected from the group consisting of ABS-295, ABS-296, ABS-298, ABS-334, ABS-357, ABS-358, ABS-359, ABS-363, ABS-368, ABS-398 and ABS-399.

3. (canceled)

4. The composition of claim 1, further comprising a pharmaceutically acceptable carrier.

5-6. (canceled)

7. A method of controlling, ameliorating or preventing shivering associated with a surgical procedure in a subject in need thereof, said method comprising the administering to said subject a pharmaceutical composition comprising at least one compound of Formula I: 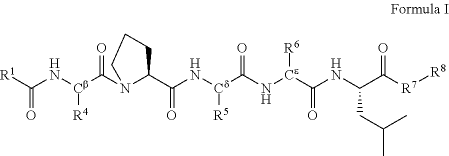
wherein:
R¹ is H, alkyl, cycloalkyl, aryl, heteroaryl, heterocyclic or C^{α}HR²R³;
R² and R⁴ are independently —(CH₂)ₘNR⁸R⁹R¹⁰, —(CH₂)ₘNR⁹C(═NR⁹)NR⁹R¹⁰, or —(CH₂)ₘ-imidazolidin-2-imin-1-yl;
R³ is —NR⁸R⁹R¹⁰, —N(R⁹)—C(—-0)R⁹, —C^{φ}HR⁹R¹⁰, —C^{φ}H(R⁹)—C(═O)R¹⁰, or —C⁴)H(C(═O)R⁹)(C(═O)R¹⁰);
R⁵ is phenyl, benzyl, —CH₂-(4-hydroxy-phenyl), —C1H-(indol-3-yl), —CH₂-(indol-4-yl), —CH₂-(napht-1-yl), —CH₂-(napht-2-yl), —CH₂-aryl, —CH₂-(heteroaryl), napht-1-yl, or napht-2-yl;
R⁶ is methyl, ethyl, propyl, isopropyl, n-butyl, i-butyl, t-butyl, (2S)-butyl, (2R)-butyl, C₅₋₆ alkyl, cyclopropyl, cyclopropylmethyl, cyclopentyl, cyclohexyl, cyclopentylmethyl, or cyclohexylmethyl;
R⁷ is —O— or —N(R⁹)—;
R⁸, R⁹ and R¹⁰ are, independently in each instance, H, alkyl, cycloalkyl, aryl, heteroaryl, heterocyclic, or (CH₂CH₂O)ₙCH₃;
m is 1, 2, 3, 4 or 5;
n is an integer of from 1 to 20;
C^{α}, C^{β}, C^{γ}, C^{ε} and C^{φ} are carbon atoms, and the stereochemistries at C^{α}, C^{β}, C^{γ}, C^{ε} and C^{φ} are independently either R or S;
formulated in a pharmaceutically acceptable salt, hydrate, pro-drug or solvate thereof.

8. The method of claim 7, wherein said at least one compound is selected from the group consisting of ABS-295, ABS-296, ABS-298, ABS-334, ABS-357, ABS-358, ABS-359, ABS-363, ABS-368, ABS-398 and ABS-399.

9. (canceled)

10. The method of claim 7, further comprising the step of administering one or more additional agents useful for treating shivering, wherein said one or more additional agents are selected from the group consisting of an opioid, an alpha-2 agonist, a serotonin neuromediator, methylphenidate, physostigmine and doxapram.

11-13. (canceled)

14. The method of claim 7, wherein said subject is feline, canine or human.

15. (canceled)

16. A method of controlling, ameliorating or preventing temperature spiking associated with a surgical procedure in a subject in need thereof, said method comprising administering to said subject a pharmaceutical composition comprising at least one compound selected from the group consisting of:
(i) a compound of Formula I: 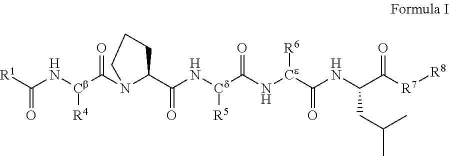
wherein:
R¹ is H, alkyl, cycloalkyl, aryl, hetcroaryl, heterocyclic or C^{α}HR²R³;
R² and R⁴ are independently —(CH₂)ₘNR⁸R⁹R¹⁰, —(CH₂)ₘNR⁹C(═NR⁹)NR⁹R¹⁰, or —(CH₂)ₘ-imidazolidin-2-imin-1-yl;
R³ is —NR⁸R⁹R¹⁰, —N(—CH₃)R⁹R¹⁰, —N(R⁹)—C(═O)R⁹, —C^{φ}HR⁹R¹⁰, —C^{φ}H(R⁹)—C(═O)R¹⁰, or —C^{φ}H(C(═O)R⁹)(C(═O)R¹⁰);
R⁵ is phenyl, benzyl, —CH₂-(4-hydroxy-phenyl), —CH₂— (indol-3-yl), —CH₂-(indol-4-yl), —CH₂-(napht-1-yl), —CH₂-(napht-2-yl), —CH₂-aryl, —CH₂-(heteroaryl), napht-1-yl, or napht-2-yl;
R⁶ is methyl, ethyl, propyl, isopropyl, n-butyl, i-butyl, t-butyl, (2S)-butyl, (2R)-butyl, C₅₋₆ alkyl, cyclopropyl, cyclopropylmethyl, cyclopentyl, cyclohexyl, cyclopentylmethyl, or cyclohexylmethyl;
R⁷ is —O— or —N(R⁹)—;
R⁸, R⁹ and R¹⁰ are, independently in each instance, H, alkyl, cycloalkyl, aryl, heteroaryl, heterocyclic, or (CH₂CH₂O)ₙCH₃;
m is 1, 2, 3, 4 or 5;
n is an integer of from 1 to 20;
C_{α}, C^{β}C^{γ}, C^{ε} and C^{φ} are carbon atoms, and the stereochemistries at C^{α}, C^{β}, C^{γ}, C^{ε} and C^{φ} are independently either R or S;
(ii) a compound of Formula II: 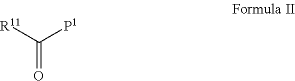
wherein
P¹ is a known or novel peptide;
R¹¹ consists of one of Formulas IIa, IIb, He and IId, wherein the N-terminus amine group of P¹ is covalently coupled to the R¹¹C(O)— group through a peptide bond, wherein:
(a) Formula IIa is: 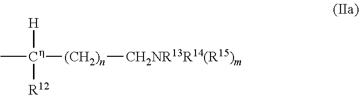
wherein
n is 0, 1, 3, 4, or 5;
m is zero or an integer of 1;
R¹² is hydrogen, a straight or branched chain alkyl group of C₁-C₁₀, a cycloalkyl group of C₃-C₆, an aromatic group of C₆″ C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination;
R¹³, R¹⁴ and R¹⁵ are, independently, hydrogen or branched or straight chain alkyl, alkenyl or alkynyl of C₁-C₁₀, an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination and with the proviso that a maximum of two of R¹³, R¹⁴ and R¹⁵ may be selected to be the aromatic, substituted aromatic, heteroaromatic or substituted heteroaromatic group; and
C^{η} is a carbon atom and the stereochemistry at C is either R or S;
(b) Formula IIb is: 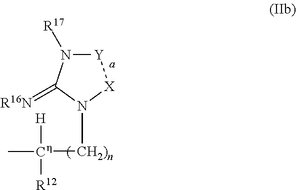
wherein
n is an integer of from 0 to 6;
when dashed line a is not present, X and Y are, independently, hydrogen or lower branched or straight chain alkyl, alkenyl or alkynyl of C₁-C₆;
when dashed line a is present, X—Y is (CH₂)_{z}, wherein z is an integer of from 1 to 8;
R¹² is hydrogen, a straight or branched chain alkyl group of C₁-C₁₀, a cycloalkyl group of C₃-C₆, an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination
R¹⁶ and R¹⁷ are independently hydrogen, lower branched or straight chain alkyl of C₁-C₁₀, lower branched or straight chain alkenyl of C₁-C₁₀, lower branched or straight chain alkynyl of C₁-C₁₀, an aromatic group of C₆-C₁₈ or a corresponding substituted aromatic group with one or two substituents selected from halogen, alkyloxy, carboxy, amide or alkyl in any combination, a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination;
C^{η} is a carbon atom and the stereochemistry at C is either R or S:
(c) Formula IIc is: 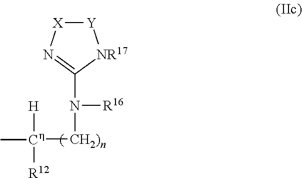
wherein
n is an integer of from 0 to
X—Y is (CH₂)_{z}, wherein z is an integer of from 0 to
R¹² is hydrogen, a straight or branched chain alkyl group of C₁-C₁₀, a cycloalkyl group of C₃-C₆, an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination;
R¹⁶ and R¹⁷ are independently hydrogen, lower branched or straight chain alkyl of C₁-C₁₀, lower branched or straight chain alkenyl of C₁-C₁₀, lower branched or straight chain alkynyl of C₁-C₁₀, an aromatic group of C₆-C₁₈ or a corresponding substituted aromatic group with one or two substituents selected from halogen, alkyloxy, carboxy, amide or alkyl in any combination, a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination;
C^{η} is a carbon atom and the stereochemistry at C is either R or S;
(d) Formula IId is: 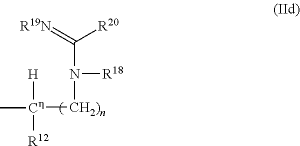
wherein
n is an integer of from 0 to 5;
R¹² is hydrogen, a straight or branched chain alkyl group of C₁-C₁₀, a cycloalkyl group of C₃-C₆, an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination;
R¹, R¹⁹, and R²⁰ are, independently, hydrogen or lower branched or straight chain alkyl, a cycloalkyl group of C₃-C₆, alkenyl or alkynyl of C₁-C₁₀, an aromatic group of C₆-C₁₈ or a corresponding substituted aromatic group with one or two substituents selected from halogen, alkyloxy, carboxy, amide or alkyl in any combination, or a heteroaromatic group of C₄-C₁₈ and one or two heteroatoms selected from oxygen, sulfur and nitrogen in any combination or a corresponding substituted heteroaromatic group with one or two substituents selected from halogen, alkyloxy, carboxy, amide or alkyl in any combination, with the proviso that a maximum of two of R¹⁸, R¹⁹, and R²⁰ may be selected to be the aromatic, substituted aromatic, heteroaromatic or substituted heteroaromatic group; and
C^{η} is a carbon atom and the stereochemistry at C is either R or S;
(iii) a neo-Trp-containing peptide selected from the group consisting of PP-1 (SEQ ID NO:26), PP-2 (SEQ ID NO:27), PP-3 (SEQ ID NO:28), PP-4 (SEQ ID NO:29), PP-5 (SEQ ID NO:30), PP-6 (SEQ ID NO:31), PP-7 (SEQ ID NO:32), PP-8 (SEQ ID NO:33), PP-9 (SEQ ID NO:34), PP-10 (SEQ ID NO:35), PP-11 (SEQ ID NO:36), PP-12 (SEQ ID NO:37), PP-13 (SEQ ID NO:38), PP-14 (SEQ ID NO:39), PP-15 (SEQ ID NO:40), PP-16 (SEQ ID NO:41), PP-17 (SEQ ID NO:42) and PP-18 (SEQ ID NO:43),
formulated in a pharmaceutically acceptable salt, hydrate, pro-drug or solvate thereof.

17. The method of claim 16, wherein said compound of Formula I is selected from the group consisting of ABS-295, ABS-296, ABS-298, ABS-334, ABS-357, ABS-358, ABS-359, ABS-363, ABS-368, ABS-398 and ABS-399.

18-24. (canceled)

25. The method of claim 16, wherein in said compound of Formula II P¹ is selected from the group consisting of H₂N-L-Arg-L-Pro-L-Tyr-L-Ile-L-Leu-COOH (SEQ ID NO:44); 1-2N-L-Arg-L-Pro-L-Tyr-L-tLeu-L-Leu-COOH (SEQ ID NO:45); H₂N-L-Lys-L-Pro-L-Tyr-L-tLeu-L-Leu-00014 (SEQ ID NO:46); H₂N-L-Lys-L-Pro-L-Trp-L-tLeu-L-Leu-COOH (SEQ ID NO:47); and H₂N-L-Arg-L-Pro-L-Trp-L-tLeu-L-Leu-COOH (SEQ ID NO:48).

26-37. (canceled)

38. The method of claim 16, further comprising administering one or more additional agents useful for treating shivering, wherein said one or more additional agents are selected from the group consisting of an opioid, an alpha-2 agonist, a serotonin neuromediator, methylphenidate, physostigmine and doxapram.

39-41. (canceled)

42. The method of claim 16, wherein said subject is human, canine or feline.

43. (canceled)

44. A method of controlling, ameliorating or preventing pain in a subject in need thereof, said method comprising administering to said subject a pharmaceutical composition comprising at least one compound of Formula I: 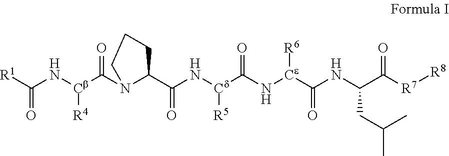
wherein:
R¹ is H, alkyl, cycloalkyl, aryl, heteroaryl, heterocyclic or C^{α}HR²R³;
R² and R⁴ are independently —(CH₂)ₘNR⁸R⁹R¹⁰, —(CH₂)ₘNR⁹C(═NR⁹)NR⁹R¹⁰, or —(CH₂)ₘ-imidazolidin-2-imin-1-yl;
R³ is —NR⁸R⁹R¹⁰, —N(R⁹)—C(═O)R⁹, —C^{φ}HR⁹R¹⁰, —OH(R⁹)—C(═O)R¹⁰, or —OH(C(═O)R⁹)(C(═O)R¹⁰);
R⁵ is phenyl, benzyl, —CH₂-(4-hydroxy-phenyl), —CH₂— (indol-3-yl), —CH₂-(indol-4-yl), —CH₂-(napht-1-yl), —CH₂-(napht-2-yl), —CH₂-aryl, —CH₂-(heteroaryl), napht-1-yl, or napht-2-yl;
R⁶ is methyl, ethyl, propyl, isopropyl, n-butyl, i-butyl, t-butyl, (2S)-butyl, (2R)-butyl, C₅₋₆ alkyl, cyclopropyl, cyclopropylmethyl, cyclopentyl, cyclohexyl, cyclopentylmethyl, or cyclohexylmethyl;
R⁷ is —O— or —N(R⁹)—;
R⁸, R⁹ and R¹⁰ are, independently in each instance, 1-1, alkyl, cycloalkyl, aryl, heteroaryl, heterocyclic, or (CH₂CH₂O)ₙCH₃;
m is 1, 2, 3, 4 or 5;
n is an integer of from 1 to 20;
C^{α}, C^{β}, C^{γ}, C^{ε} and are carbon atoms, and the stereochemistries at C^{α}, C^{β}, C^{γ}, C^{ε} and C^{φ} are independently either R or S;
formulated in a pharmaceutically acceptable salt, hydrate, pro-drug or solvate thereof.

45. The method of claim 44, wherein said at least one compound is selected from the group consisting of ABS-295, ABS-296, ABS-298, ABS-334, ABS-357, ABS-358, ABS-359, ABS-363, ABS-368, ABS-398 and ABS-399.

46-48. (canceled)

49. A method of controlling, ameliorating or preventing psychosis in a subject in need thereof, said method comprising administering to said subject a pharmaceutical composition comprising at lease one compound of Formula I: 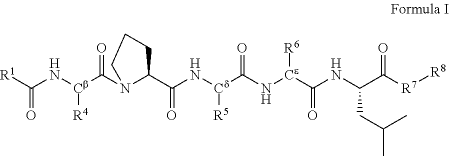
wherein:
R¹ is H, alkyl, cycloalkyl, aryl, heteroaryl, heterocyclic or C^{α}HR²R³;
R² and R⁴ are independently —(CH₂)ₘNR⁸R⁹R¹⁰, —(CH₂)ₘNR⁹C(═NR⁹)NR⁹R¹⁰, or —(CH₂)ₘ-imidazolidin-2-imin-1-yl;
R³ is —NR⁸R⁹R¹⁰, —N(R⁹)—C(═O)R⁹, —C^{φ}HR⁹R¹⁰, —C^{φ}H(R⁹)—C(═O)R¹⁰, or —C^{φ}H(C(═O)R⁹)(C(═O)R¹⁰);
R⁵ is phenyl, benzyl, —CH₂-(4-hydroxy-phenyl), —CH₂— (indol-3-yl), —CH₂-(indol-4-yl), —CH₂-(napht-1-yl), —CH₂-(napht-2-yl), —CH₂-aryl, —CH₂-(heteroaryl), napht-1-yl, or napht-2-yl;
R⁶ is methyl, ethyl, propyl, isopropyl, n-butyl, i-butyl, t-butyl, (2S)-butyl, (2R)-butyl, C₅₋₆ alkyl, cyclopropyl, cyclopropylmethyl, cyclopentyl, cyclohexyl; cyclopentylmethyl, or cyclohexylmethyl;
R⁷ is —O— or —N(R⁹)—;
R⁸, R⁹ and R¹⁰ are, independently in each instance, H, alkyl, cycloalkyl, aryl, heteroaryl, heterocyclic, or (CH₂CH₂O)ₙCH₃;
m is 1, 2, 3, 4 or 5;
n is an integer of from 1 to 20;
C^{α}, C^{β}, C^{γ}, C^{ε} and C^{φ} are carbon atoms, and the stereochemistries at C^{α}, C^{β}, C^{γ}, C^{ε} and C^{φ} are independently either R or S;
formulated in a pharmaceutically acceptable salt, hydrate, pro-drug or solvate thereof.

50. The method of claim 49, wherein said at least one compound is selected from the group consisting of ABS-295, ABS-296, ABS-298, ABS-334, ABS-357, ABS-358, ABS-359, ABS-363, ABS-368, ABS-398 and ABS-399.

51-53. (canceled)

54. A method of lowering the body temperature of a subject in need thereof, said method comprising administering to said subject a pharmaceutical composition comprising at least one compound of Formula I: 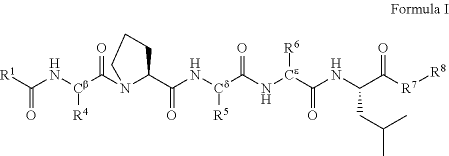
wherein:
R¹ is H, alkyl, cycloalkyl, aryl, heteroaryl, heterocyclic or C^{α}HR²R³;
R² and R⁴ are independently —(CH₂)ₘNR⁸R⁹R¹⁰, —(CH₂)ₘNR⁹C(═NR⁹)NR⁹R¹⁰, or —(CH₂)ₘ-imidazolidin-2-imin-1-yl;
R³ is —NR⁸R⁹R¹⁰, —N(R⁹)—C(═O)R⁹, —C^{φ}HR⁹R¹⁰, —OH(R⁹)—C(═O)R¹⁰, or —C^{φ}H(C(═O)R⁹)(C(═O)R¹⁰);
R⁵ is phenyl, benzyl, —CH₂-(4-hydroxy-phenyl), —CH₂— (indol-3-yl), —CH₂-(indol-4-yl), —CH₂-(napht-1-yl), —CH₂-(napht-2-yl), —CH₂-aryl, —CH₂-(heteroaryl), napht-1-yl, or napht-2-yl;
R⁶ is methyl, ethyl, propyl, isopropyl, n-butyl, i-butyl, t-butyl, (2S)-butyl, (2R)-butyl, C₅₋₆ alkyl, cyclopropyl, cyclopropylmethyl, cyclopentyl, cyclohexyl, cyclopentylmethyl, or cyclohexylmethyl;
R⁷ is —O— or —N(R⁹)—;
R⁸, R⁹ and R¹⁰ are, independently in each instance, H, alkyl, cycloalkyl, aryl, heteroaryl, heterocyclic, or (CH₂CH₂O)ₙCH₃;
m is 1, 2, 3, 4 or 5;
n is an integer of from 1 to 20;
C^{α}, C^{β}, C^{γ}, C^{ε} and C^{φ} are carbon atoms, and the stereochemistries at C^{α}, C^{β}, C^{γ}, C^{ε} and C^{φ} are independently either R or S;
formulated in a pharmaceutically acceptable salt, hydrate, pro-drug or solvate thereof.

55. The method of claim 54, wherein said at least one compound is selected from the group consisting of ABS-295, ABS-296, ABS-298, ABS-334, ABS-357, ABS-358, ABS-359, ABS-363, ABS-368, ABS-398 and ABS-399.

56-58. (canceled)
